(19) United States
(12) Patent Application Publication
(10) Pub. No.: US 2003/0138812 A1
(43) Pub. Date: Jul. 24, 2003

(54) Assessment of ecosystem health by evaluating multiple biomarkers in a nonhuman organism

(76) Inventor: Craig A. Downs, Mansfield, OH (US)

Correspondence Address: REED & ASSOCIATES, 800 MENLO AVENUE, SUITE 210, MENLO PARK, CA, 94025, US

(21) Appl. No.: 10/251,093

(22) Filed: Sep. 20, 2002

Related U.S. Application Data
(63) Continuation of application No. 09/662,187, filed on Sep. 15, 2000.

(51) Int. Cl. A61K 49/00; A61K 31/00; A01N 61/00; C12Q 1/70; C12Q 1/68

(52) U.S. Cl. 435/6; 514/1; 424/9.2; 435/5

(57) ABSTRACT

A novel method for assessing the health of an ecosystem is provided. The method comprises measuring the levels of a plurality of physiological parameters of a nonhuman organism living in the ecosystem and determining whether the organism is healthy or physiologically stressed based on the levels of the tested physiological parameters. To the extent that the organism is physiologically stressed, the results of the measurement are employed to assess the type and degree of stress occurring in the ecosystem.

### RELATED APPLICATIONS

This application is a continuation of U.S. patent application Ser. No. 09/662,187, filed Sep. 15, 2000.

### TECHNICAL FIELD

This invention relates generally to a method for assessing the health of an ecosystem, and more particularly relates to a method for assessing the health of an ecosystem by evaluating multiple biomarkers of a nonhuman organism in the ecosystem. The invention additionally relates to a method for identifying specific stressors, e.g., heat, light, chemical contaminants, etc., that have an impact on an ecosystem and living organisms therein. Further, the invention relates to a method for using the aforementioned information to predict the long-term effect of the identified stressors on the ecosystem.

### BACKGROUND

Environmental and anthropogenic pressures often decrease the health and stability of ecosystems, but the precise effects of these stressors remain largely unknown. Most ecosystem health assessment focuses on either the abiotic components of an ecosystem (i.e., contaminant analysis) or ecological responses (i.e., species richness and population density) because methods to monitor these parameters are well developed (e.g., Otte et al. (1998), “Relation Between Heavy Metal Concentrations in Salt Marsh Plants and Soil,” Environmental Pollution 82:13-22; Wilson et al. (1996), “Measuring and Monitoring Biological Diversity: Standard Methods for Mammals” (Smithsonian Institution Press, Washington, D.C.). Technological advances that would reveal how the biota respond to environmental stressors have been unavailable. This creates a gap in our knowledge of how stressors affect ecosystems, which are complex and biologically hierarchical systems (O'Connor et al. (1996), “Toward the Incorporation of Spatiotemporal Dynamics into Ecotoxicology,” in Population Dynamics in Ecological Space and Time, Rhodes et al., eds. (University of Chicago Press, Chicago, Ill.) at pp. 281-317). For example, although traditional water and sediment analyses can document the quality and quantity of a contaminant in the environment, these analyses cannot readily describe (and therefore cannot predict) biotic responses to that contaminant. The presence of a particular contaminant does not necessarily decrease ecosystem health, because compensatory mechanisms operating at different hierarchical levels (e.g., cell, tissue, individual levels) may ameliorate stress before it reduces the fitness of an individual organism, or alters its functional role in the community. Population and ecological responses only assess a part of the hierarchy of an ecosystem. Monitoring species diversity or population density only measures the cumulative indirect effects of most stressors on an ecosystem. This is because under most circumstances, a stressor indirectly affects higher levels of the ecosystem hierarchy but directly affects molecular and cellular level processes. What is needed is a technology that can rapidly detect and identify the effects of different stressors on the cellular and molecular processes that govern organismal health and fitness in complex ecosystems. Cellular or molecular biomarkers are an important means for assessing those processes.

Biomarkers are biological parameters that reflect changes in biological systems that indicate their condition or health. The major obstacle in the productive use of molecular biomarkers has been the need to establish a rational and defined conceptual system for their use and interpretation. Numerous studies demonstrate that a particular molecular biomarker displays a significant response when a specific organism is exposed to a specific stressor. Missing from many biomarker studies is an interpretation of the response on a cellular physiological level, and understanding of how that response is linked to organismal health. Prior to the present invention, no one has documented systematic use of multiple biomarkers in combination to assess the health condition of a nonhuman, nonagricultural organism to evaluate the health of a complex ecosystem.

The present invention provides an integrated molecular biomarker system that reflects responses of known cellular and physiological processes. The system diagnoses organismal health by (1) quantifying parameters that reflect cellular and physiological conditions, and (2) providing evidence for identification of certain classes of stressors that are affecting cellular responses of the organism. The method involves assaying critical components of the cellular superstructure and the status of general properties of cellular metabolism. The biochemical and cellular processes monitored are common to all organisms, from insects and mammals to bacteria and plants. The array of molecular biomarkers used is tailored to the specific species and likely sources of organismal stress. For example, free proline is an excellent biomarker in plant species because it accumulates in response to metabolic stress and protects the NAD:NADH redox potential of plant mitochondria. In animals, proline does not have the same function and therefore is an inappropriate molecular biomarker. Thus, a thorough knowledge of the stress physiology and cellular biology of the organism is an essential criterion for choosing an appropriate molecular biomarker array.

A primary objective of an environmental monitoring or ecosystem health assessment project is to determine the overall health of selected population(s) in a particular habitat. Accomplishing this requires defining nominal levels of a particular molecular biomarker(s) for that species. Nominal levels are defined statistically and must be inferred by both laboratory and field studies for that species, although the limitation of each stratagem must be taken into account when making inferences concerning deviations from the nominal level. If a disease state does exist in a particular population in response to a particular stressor, not only can a diagnosis be made concerning the health of the population, but models can be developed and used to predict the outcome of the population in response to the stressor. If verified in the field (ecoepidemiology), these models can be used by environmental managers to ameliorate, mitigate, or manage an environmental condition (Bro-Rasmussen et al. (1984), “Ecoepidemiology—A Casuistic Discipline Describing Ecological Disturbances and Damages in Relation to Their Specific Causes: Exemplified by Chlorinated Phenols and Chlorophenoxy Acids,” Regul. Toxicol. Pharmacol. 4:391-399). Prediction of ecological status would be one of the most powerful tools for environmental managers for both rehabilitating a stressed ecosystem and preventing further adverse effects from an identified stressor. Depending on the species, ecosystem, and type of stress, the molecular biomarker system of the invention can predict physiological and ecological outcomes as a result of a specific stressor.

The greatest challenge in environmental monitoring and ecosystem health assessment is determining the overall health status or condition of an ecosystem. A number of significant problems arise when inferring the health condition of an ecosystem from the status of a single species. For example, increases in population density or biomass of a primary producer may not be representative of the condition of other trophic levels in that ecosystem, and thus is an inaccurate estimate of ecosystem health (Culotta et al. (1995), “A Physioloigcal Role for Saccharomyces Cerevisiae Cu/Zn Superoxide Dismutase in Copper Buffering,” J. Biol. Chem. 270(50):29991-7). A number of classical ecological studies demonstrate that increases or improvements in the condition of one trophic level (e.g., primary producer), may arise from adverse or stressed conditions affecting other trophic levels (e.g., herbivores) (see, for example, Bothwell et al. (1994) “Ecosystem Response to Solar Ultraviolet-B Radiation: Influence of Trophic-Level Interactions,” Science 265:97-100).

To overcome these obstacles, the present invention provides a new system that substantially improves the accuracy and specificity of an ecosystem health assessment, allowing identification of probable stressors, and, depending on the quality and the degree of stress, can predict the outcome of a population or an entire ecosystem in response to a stressed condition.

### SUMMARY OF THE INVENTION

It is thus a primary object of the invention to provide a novel method for assessing the health of an ecosystem that addresses the above-described needs in the art.

It is another object of the invention to provide a method for assessing the future health of an ecosystem.

It is yet another object of the invention to provide a method for assessing the health of an ecosystem that comprises identifying at least one stressor in the ecosystem that is causing the organism to be physiologically stressed.

Additional objects, advantages and novel features of the invention will be set forth in part in the description that follows, and in part will become apparent to those skilled in the art upon examination of the following, or may be learned by practice of the invention.

In one embodiment, a method is provided for assessing the health of an ecosystem comprising a series of steps. The first step requires measuring the levels of a plurality of physiological parameters of a nonhuman organism living in the ecosystem. Each of these physiological parameters corresponds to a single and specific cellular function. The next step requires identifying as normal or abnormal the levels of each of the physiological parameters measured in the first step. In this context, a normal level is within a range associated with a healthy organism and an abnormal level is within a range associated with a physiologically stressed organism. The results are then employed, in a third step, to determine whether the organism is healthy or physiologically stressed. To the extent that the organism is physiologically stressed, the results of the second step are then used to assess the type and degree of stress occurring in the ecosystem. Finally, the invention provides a further step that includes predicting the future health of the ecosystem based on the physiological impact of the stress on the organism.

The invention also provides the ability to identify the stressor or stressors in the ecosystem that are causing the organism to be physiologically stressed. Such stressors include, for example, heat, light, and chemical contamination (e.g., heavy metal, polyaromatic hydrocarbon, organic solvent and/or herbicide contamination).

The measuring step must be carried out by an assay that can quantify the desired molecular biomarker, i.e., the physiological parameter, in the tested organism. Preferred assays include centrifugation, chromatography, electrophoresis, enzyme immunoassay (EIA), immunoprecipitation, passive agglutination and solid phase affinity assays. A particularly preferred assay for measuring the molecular biomarker is an enzyme immunoassay, e.g., ELISA or ELISPOT.

### DETAILED DESCRIPTION OF THE INVENTION

I. DEFINITIONS AND OVERVIEW:

Before describing the present invention in detail, it is to be understood that this invention is not limited to specific reagents, assays formats, or the like, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise.

In describing and claiming the present invention, the following terminology will be used in accordance with the definitions set out below.

As used herein, an “ecosystem” is a system formed by the interaction of a community of organisms among themselves and with their physical environment. An “ecosystem” represents the totality of a multitude of individual factors, both organic and inorganic. Inorganic factors that influence any given ecosystem include energy factors, e.g., light and thermal energy, and chemical factors, e.g., soil composition and water quality. Organic factors of an ecosystem include the community of species living in the system. In turn, each species has its own hierarchical arrangement of complexity that may influence, or be influenced by, the surrounding ecosystem. In descending order of complexity, each species comprises levels of population, individual, organ, tissue and cell. Each level represents an important aspect of the overall ecosystem.

As used herein, a “biomarker” refers to a physiological parameter of an organism that can provide a measurable change when the organism is stressed. Generally, the physiological parameter that is measured is a biological molecule and may be referred to as a “molecular biomarker.” Thus, the amount of any particular “biomarker” or “molecular biomarker” will change (i.e., increase or decrease) depending on the degree and type of stress. By measuring one or more “biomarkers” or “molecular biomarkers,” it is possible to determine the health of the organism and, in turn, the health of the entire ecosystem.

A “stressor” as used herein refers to an agent, condition or other stimulus that causes stress, i.e., a change in homeostasis, to particular system. Generally, a “stressor” within any particular ecosystem has a direct impact on an organism at the cellular level, and may, in turn, indirectly affect a higher organic level (e.g., tissue, organ, individual, population) of the organism within the ecosystem.

“Normal,” as in a “normal level” and “nominal,” as in a “nominal level” of a particular physiological parameter or molecular biomarker, refers to a value that is associated with a healthy organism. Generally, a range of “normal levels” can be established for a particular physiological parameter for a specific organism by conventional experimentation and statistical analysis. Thus, a “normal level” of any physiological parameter (or molecular biomarker) is a value found within the range of healthy values established for that physiological parameter (or molecular biomarker) for that particular organism.

The term “abnormal,” as in an “abnormal level” of a particular physiological parameter or molecular biomarker, refers to a value outside the range established as normal. Thus, an “abnormal level” may be higher or lower than the range established as normal. An “abnormal level” is associated with an organism that is stressed.

“Optional” or “optionally” means that the subsequently described circumstance may or may not occur, so that the description includes instances where the circumstance does occur and instances where it does not.

In one embodiment, then, the invention pertains to a method for assessing the health of an ecosystem. Initially, the levels of a plurality of physiological parameters or molecular biomarkers are determined for a nonhuman organism living in the ecosystem of interest. Each physiological parameter corresponds to a specific cellular function. Determing the particular physiological parameters to measure and establishing a range of normal values for each parameter is discussed supra. The next step requires identifying as either normal or abnormal the levels of the measured physiological parameters. A third step requires determining from the results of the previous step whether the organism is healthy or physiologically stressed. An abnormal level of a physiologic parameter tends to indicate that the organism is experiencing stress. Conversely, normal levels of a physiologic parameter is generally indicative of an organism that is free from stress. After determining the normalcy/abnormalcy of a series of physiologic parameters, it is possible to determine whether the organism is healthy or physiologically stressed. To the extent that the organism is physiologically stressed, a fourth step requires using the results of the second step to assess the type and degree of stress occurring in the ecosystem. Finally, the invention provides a further step that includes predicting the future health of the ecosystem based on the physiological impact of the stress on the organism.

II. THE ORGANISM:

The method of the invention can be adapted such that almost any nonhuman organism found in the ecosystem can provide the physiological parameters or molecular biomarkers that are tested. In addition to being nonhuman, however, the organism should also be nonagricultural. Selection of a specific organism is based on a variety of factors including ecological importance, abundance (i.e., percentage of biomass in the ecosystem being studied), fidelity of the site, economic importance, knowledge of the biochemistry and physiology of the species (e.g., tendency to bioaccumulate toxins, resistance and susceptibility to stress), and ease of sample acquisition, preparation and analysis.

Preferred organisms for use in the method include species from the groups consisting of coral, plant (i.e., nonagricultural plant species), mollusc (including gastropods and bivalves), crustacean (e.g, shrimp, crabs and barnacles), vertebrate (nonhuman), bacterial, viral, multicellular species from the Protoctista kingdom and protozoan organisms. Although any coral species from the Coelenterata phylum may be used, coral species from the Anthozoa and Hydrozoa classes are preferred. Preferred subclasses of the Anthozoa class include the Ceriantipatharia, Octocorallia and Zoantharia subclasses. Preferred orders of the Zoantharia subclass include the Actiniaria, Scleractinia and Zoanthidea orders, while a preferred order of the Hydrozoa class is the Milleporina order. A particularly preferred coral for use in the present method is Montastraea faveolata.

Any nonagricultural, i.e., not actively cultivated, plant species may also be employed in the method. A particularly preferred taxonomic class of plants is the Liliopsida class of plants. A preferred order of the Liliopsida class is the Cyperales order. Furthermore, among the Cyperales order, the Poaceae family (commonly referred to as the grass family) is preferred. Spartina alternaflora (saltmarsh cordgrasses) is a particularly preferred species.

Any member of the Mollusca phylum may be employed in the method. Particularly preferred classes of the Mollusca phylum include the Gastropoda (limpets and snails), Bivalvia (clams and cockles) and Mesogastropoda classes. Preferred orders of the Bivalvia class include the Mytiloida (mussels) and Osteroida (scallops and oysters) orders. Within the Mesogastropoda (littorines) class, a preferred superfamily is the Littorinacea superfamily. Within the Gastropoda class, species from the Pulmonata subclass are preferred with species from the Stylommatophora order being particularly preferred. Other preferred orders within the Gastropoda class include the Archaeogastropoda (limpets) and Neogastropoda (whelks) orders. Ilyanassa obsoleta is a specific genus and species of gastropod that is particularly preferred.

Although any species from the Arthropoda phylum (i.e., the arthropods) may be used as the organism for the present method, the Crustacea subphylum is preferred. Within the subphylum Crustacea, organisms from the Branchiopoda, Cirripedia or Malacostraca classes are particularly preferred. Preferred orders of the Brachiopoda class include the Anostraca, Conchostraca and Notostraca orders. A preferred subclass of the Malacostraca class is the Eumalacostraca subclass while preferred superorders of the Eumalacostraca subclass include the Eucaridia and Hoplocarida superorders. Within the Eucarida superorder, the Decapoda order (e.g., crabs, lobsters and shrimp) is preferred with the Natantia suborder being particularly preferred. The Brachyura is a preferred infraorder of the suborder Natantia. A preferred order of the Hoplocarida superorder is the Stomatopoda order. The Palaeomonetes pugio is a specific genus and species that is particularly preferred.

Although any nonhuman vertebrate (i.e., from the Chordata phylum, Vertebrata subphylum) may be used as the organism in the method, preferred vertebrate classes include the Osteichthyes (bony fishes), Amphibia, Reptilia, Aves (birds), and Mammalia classes. Preferred Osteichthyes orders include the Salmoniformes, Gasterosteiformes and Perciformes orders. Within the Gasterosteiformes order, a preferred family includes the Sungnathidae (pipefishes and seashorses) family. Within the Perciformes order, preferred families include the Haemulidae (grunts) and Pomacentridae (damselfishes) families. A particularly preferred fish species is Fundulus heteroclitus (mummichog). Preferred Amphibia orders include Anura (frogs), Caudata (salamanders), and Gymnophiona (the caecilian) orders. Preferred Reptilia orders include Testudines (turtels), Squamata (lizards and snakes), and Crocodylia (crocodilians) orders.

Preferred Mammalia orders include the Cetacea and Carnivora Orders. Preferred suborders for the Cetacea order include the Mysticeta (baleen whales) and Odontoceta (toothed whales) suborders. Preferred suborders for the Carnivora order include Caniformia (dogs, mustelids, procyonids and pinnipeds). Preferred mammalian superfamilies include Otariidae (sea lions, fur seals and walruses), Musteloidae (otters and badgers), Phocidae (hair seals or earless seals), and Sirenia (dugongs, sea cows and manatees) superfamilies. Preferred manatees include the Trichechidae inunguis (Amazonian manatee), Trichechus manatus (West Indian Manatee) and Trichechus senegalensis (West African manatee).

Although any bacterial species may be used in the present method, a preferred class is the Cyanophyceae class, preferably from the Cyanophanaceae (cyanobacteria) family. A preferred order of bacteria includes the Cytophagales order, preferably from the Beggiataceae, Pseudomonadaceae, Enterobacteriaceae or Vibrionaceae families. Plant viruses and invertebrate virus are preferred viral organisms for use in the method. Multicellular organisms from the Protoctista kingdom may be used in the present method. Preferred phyla from this kingdom include Chrysophyta (diatoms), Pyrrophyta (dinoflagellates), and Phaeophyta (brown algae). Preferred dinoflagellates include those from the genus Symbiodinium, e.g., coral symbiotic dinoflagellate and zooxanthaellae species. Protozoans may also be used in the present method. Protozoans from the Granuloreticulosa class are preferred organisms for use in the system. A preferred order of the Granuloreticulosa class is the Foraminferida order.

III. TYPES OF STRESSORS:

Any stress that elicits a detectable physiological change in an organism can be detected in accordance with the present method. For example, the stressor may originate from natural sources, e.g., periodic increases in water temperature due to changes in warm ocean currents. Alternatively, the stressor may originate from human sources and include, for example, chemical pollutants (e.g., insecticides, herbicides, diesel fuel, bunker fuel or heavy metals, etc.), human waste or effluent from a power plant.

Stressors for which the present method is particularly well-suited to detect include, light stress, heat stress, oxidative stress, fuel stress (e.g., stress derived from exposure to the water accommodating fractions of diesel fuel and/or #2 bunker fuel), heavy metal stress (e.g., cadmium chloride), herbicide stress (e.g., atrazine), and pesticide stress (e.g., endosulfan). As will be recognized, the method can be adapted to detect a variety of different stressors.

IV. PHYSIOLOGIC PARAMETERS (BIOMARKERS):

A battery of biomarkers is used to test the overall health of the individual organism which, in turn, is used to assess the overall health of the ecosystem. Each biomarker in the array must be tailored to the specific organism and the stress being tested. As previously indicated, free proline is an excellent biomarker or physiological parameter in plant species because it accumulates in response to metabolic stress and protects the NAD:NADH redox potential of plant mitochondria. In animals, however, proline does not have the same function and therefore is an inappropriate molecular biomarker for animal species. Similarly, B-crystallin (a small heat-shock protein) is generally present only in the cytosol of an animal that is experiencing stress. Consequently, this biomarker is inappropriate in plant species. Only after an organism's biochemistry and physiology are fully understood is it possible to identify the biomarkers in an organism that can be employed in carrying out the method of the invention. A description of some of the preferred biomarkers is provided below.

P-glycoprotein (P-gp):

P-glycoprotein plays a role in xenobiotic detoxification. Currently, it is believed that P-glycoprotein effectively processes certain xenobiotics to exit the cell. Sustained exposure to certain xenobiotics causes an increase in the cellular level of P-glycoprotein. Animals deficient in P-glycoproteins are viable and do not exhibit any obvious abnormalities. The pharmacokinetic activity and relative toxicity of several compounds are, however, altered in these P-glycoprotein-deficient animals. For example, P-glycoprotein in mammals plays a significant role in preventing certain xenobiotics from crossing the blood-brain barrier: a decrease in P-glycoprotein would result in an increase in the amount of xenobiotics reaching the brain. P-glycoproteins are members of a superfamily of proteins called the adenosine triphosphate (ATP) binding cassette that act as channels and transporters of solutes across membranes. Induction of certain P-glycoproteins indicates a response to a xenobiotic exposure. See generally Ueda et al. (1999), “Comparative Aspects of the Function and Mechanism of SUR1 and MDR1 Proteins,” Biochem. Biophys. Acta 1461:305-313 and Borst et al. (1999), “The Multidrug Resistance Protein Family,” Biochem. Biophys. Acta 1461:347-357.

Dehydrin:

Dehydrins are proteins synthesized in photosynthetic organisms (e.g., cyanobacteria, angiosperms, etc.) in response to a number of specific environmental and hormonal conditions. Depending on the particular isoform, dehydrins can be induced by cold stress, salt-stress or osmotic stress. Some isoforms are induced by absisic acid (a plant stress hormone). There are a number of dehydrin isoforms, each isoform localizing to a certain cellular locality. For example, a number of dehydrins associate with the plasma membrane, while others associate with the thylakoid membrane of chloroplasts, and still others associate with the inner mitochondrial membrane. In plant mitochondria, dehydrin isoforms DL-45 and DL-55 are both known to associate in a multimeric structure and uncouple oxidative phosphorylation during osmotic and cold stress, thereby reducing the inadvertent production of reactive oxygen species during these stresses. Dehydrins are not present during normal, mature physiological conditions but accumulate in response to certain environmental stresses in addition to developmental responses during seed development. See generally Ismail et al. (1999), “Allelic Variation of a Dehydrin Gene Cosegregates with a Chilling Tolerance During Seedling Emergence,” Proc. Natl. Acad. Sci. USA 96:13566-13577, Borovskii et al. (2000), “Accumulation of Protiens Immunochemically Related to Dehydrins in Mitochondria of Plants Exposed to Low Temperature,” Dokl. Biochem. 371:46-49.

Glutathione (GSH):

Glutathione is a tripeptide with a single cysteine residue and plays a significant role in xenobiotic detoxification and as an antioxidant. Total glutathione content is an important marker for cellular redox potential. Sies et al. (1999), “Glutathione and its Role in Cellular Functions,” Free Radic. Biol. Med. 27:916-921; Asada et al. (1988), “Metabolism and Function of Glutathione in Plants,” Tanapakushitsu Kakusan Koso 33:1513-1421; Halliwell (1999), “Antioxidant Defense Mechanism: From the Beginning to the End (of the Beginning),” Free Radic. Res. 31:261-272; Sagara et al. (1998), “Cellular Mechanism of Resistance to Chronic Oxidative Stress.” Free Radic. Biol. Med. 24:1375-1389. During oxidative stress, reduced glutathione (GSH) acts as an anti-oxidant on several different pathways: (1) GSH reacts with hydrogen peroxide via glutathione peroxidase to form water and oxidized glutathione (GSSG); (2) in conjunction with ascorbate, glutathione is an essential component of the Asada-Halliwell pathway—a major anti-oxidant cyclic pathway; and (3) GSH can act independently as a hydroxyl and superoxide quencher. Within hours of an initial exposure to oxidative stress, intracellular GSH levels decrease significantly. However, as a compensatory action, GSH levels can later increase several-fold, compared to levels prior to the oxidative stress event.

Lipid Peroxide (LPO):

Lipid radicals, especially those derived from polyunsaturated fatty acids, may react with active oxygen species to form peroxyl adducts, which in turn may react with other lipids in an autooxidation chain reaction. Armstrong et al. (1994), “The Analysis of Free Radicals, Lipid Peroxides, Antioxidant Enzymes and Compounds related to Oxidative Stress as Applied to the Clinical Chemistry Laboratory,” Adv. Exp. Med. Biol. 366:43-58; Girotti (1998), “Lipid Hydroperoxide Generation, Turnover, and Effector Action in Biological Systems,” J. Lipid Res. 39:1529-1542; Duthie (1993), “Lipid Peroxidation,” Eur. J Clin. Nutr. 47:759-764; Rilkans et al. (1997), “Lipid Peroxidation, Antioxidant Protection and Aging,” Biochim. Biophys Acta 1362:16-127; Halliwell et al. (1999), “Antioxidant Defense Mechanism: from the Beginning to the End (of the Beginning,” Free Radic. Res. 31:261-272. Formation of lipid peroxides is an indication that the integrity of biological membranes is being assaulted or has been compromised. Therefore, LPO reflects the structural integrity of one component of the cell superstructure. Production of LPO also indicates that active oxygen species are overwhelming a number of different antioxidant species. LPO, thus, also gauges oxidative stress.

Ubiquitin:

Ubiquitin is a 76-residue protein found in most phyla, which marks proteins for rapid degradation. Ubiquinated proteins are then degraded by proteolytic enzymes known as proteosomes. Hershko et al. (1998), “The Ubiquitin System,” Annu. Rev. Biochem. 67:425-479; Iwai (1999), “Roles of the Ubiquitin System in Stress Response,” Tanpakushitsu Kakusan Kuso 44: 759-765; Goff et al. (1988), “Protein Breakdown and the Heat-Shock Response,” Ubiquitin (ed. M. Rechsteiner), New York, N.Y.: Plenum Press, pp. 207-238; Jennissen (1995), “Ubiquitin and the Enigma of Intracellular Protein Degredation,” Eur. J. Biochem. 231:1-30; Mimnaugh et al. (1999), “The Measurement of Ubiquitin and Ubiquinated Proteins,” Electrophoresis 20:418-428; Hawkins (1991), “Protein Turnover: A Functional Appraisal,” Funct. Ecol. 5:222-233. During stress, proteins are targeted for degradation, usually because they have become irreversibly denatured. Increased ubiquitin levels indicate higher levels of protein degradation, and thus, increased protein turnover. To compensate for the lower levels of functional protein caused by stress, cells increase production of these same proteins. Consequently, ubiquitin reflects the structural integrity of the protein component of the cell superstructure. Increased ubiquitin levels: (1) indicate a protein-denaturing stress is occurring, (2) indicate an increased expenditure of energy (required to compensate for stress-induced protein turnover), and (3) compared to baseline levels for a particular species, may act as an indicator of individual fitness (Hawkins (1991), supra).

Metallothioneins:

Metallothioneins are cysteine-rich, low molecular weight proteins that bind a variety of metals depending on the class of metallothionein; see Klassen et al. (1999), “Metallothionein: An Intracellular Protein to Protect Against Cadmium Toxicity,” Annu. Rev. Pharmacol. Toxicol. 39:267-294. In vitro evidence demonstrates that metallothioneins may act as an anti-oxidant, a factor to ensure a reservoir of zinc, and can protect against metal toxicity. In vivo evidence using transgenic animals shows strong support for the function of metallothioneins as a protectant against metal toxicity. Metallothioneins can be grouped into three classes, commonly referred to as metallothionein I (MTI), metallothionein II (MTII), and metallothionein III (MTIII). (Klassen et al., supra). Metallothioneins are strongly induced by metal toxicity and are indicators of metal exposure.

Heat Shock Proteins Hsp60 and Hsp70:

Heat shock proteins Hsp60 and Hsp70 are molecular chapererones that regulate protein structure and function under normal physiological conditions, as well as during stress. Both Hsp60 and Hsp70 are ubiquitous chaperones found in all phyla, and are essential components for cellular function during both normal and stressed conditions. Hartl (1996), “Molecular Chaperones in Cellular Protein Folding,” Nature 381:571-579. Chaperones renature denatured proteins into active states in an ATP-dependent manner. Furthermore, mitochondrial and chloroplastic Hsp60 and Hsp70 homologues work in concert—an essential multi-step pathway for correct conformation of protein structure. Hsp60 and Hsp70 levels increase in response to stress, specifically in response to increased protein synthesis and denaturation. These two chaperones indicate that the cell's “house-keeping” proteins are experiencing denaturing conditions. Thus, measurement of these chaperones is an index of the structural integrity of the protein component of the cell superstructure.

Small Heat-Shock Proteins B-Crystallin, chlpsHsp, and sHsps:

Small heat-shock proteins B-crystallin, chlpsHsp, and sHsps all share a certain level of homology with one another, but have different cellular functions (de Jong et al. (1993), “Evolution of the Alpha-Crystallin/Small Heat-Shock Protein Family,” Mol. Biol. Evol. 10: 103-126). The small heat-shock proteins from all phyla share a common motif near the carboxyl terminus of the protein, known as the “heat-shock domain” or -crystallin domain. Other areas of these proteins are not homologous and are specific to the subfamily of sHsps. For example, the chloroplast sHsp has a unique forty-residue domain known as the methionine-rich domain, which is evolutionarily conserved from species that have diverged over 500 million years ago (Downs et al. (1998), “The Mitochondrial Small Heat-Shock Protein Protects NADH:Ubiquinone Oxidoreductase of the Electron Transport Chain During Heat Stress in Plants,” FEBS Letters 430:246-250).

In most cases, the small heat-shock proteins (sHsps) are absent under optimal growing conditions and are only elicited in response to stress (de Jong et al. (1993, supra). B-crystallin is a small heat-shock protein found only in the cytosol of animals, where it protects cytoskeletal elements during stress (Derham et al. (1999), “-Crystallin as a Molecular Chaperone,” Prog. Retin. Eye Res. 4:463-509). The chlpsHsp is a small heat-shock protein present only in the chloroplast, where it specifically associates with the Oxygen Evolving Complex of Photosystem II (Downs et al. (1999), “The Chloroplast 22-kD Heat-Shock Protein: A Lumenal Protein That Associates With the Oxygen Evolving Complex and Protects Photosystem II During Heat Stress,” J. Plant Physiol. 155:477-487; Heckathorn et al. (1998), “The Small Methionine-Rich Chloroplast Heat-Shock Protein Protects Photosystem II Electron Transport During Heat Stress,” Plant Physiol. 116:439-444). The chlpsHsp protects Photosystem II activity during heat stress, ultraviolet radiation exposure, and oxidative stress, most likely via a recycling antioxidant mechanism (Downs et al. (1999), “The Chloroplast Small Heat-Shock Protein: Evidence For a General Role in Protecting Photosystem II Against Oxidative Stress and Photoinhibition,” J. Plant Physiol. 55:488-496). Finally, the plant sHsp determines the concentration of four classes of plant sHsps: two cytosolic sHsp classes, the plant mitochondrial sHsp, and the chloroplast sHsp. Both the mitochondrial (plant, fungal, and animal) and chloroplast sHsps act to protect the cell's energy metabolism during stress, oxidative phosphorylation or photosynthesis (Downs et al. (1999), “Evidence For a Novel Set of Small Heat-Shock Proteins That Associates With the Mitochondria of Murices PC 12 Cells and Protects NADH:Ubiquinone Oxidoreductase From Heat and Oxidative Stress,” Arch. Biochem. Biophys. 365:344-350). Hence, the presence and concentration of these different small heat-shock proteins provides an index for the physiological status of several different metabolic pathways in the cell.

Copper/Zinc and Manganese Superoxide Dismutases (Cu/Zn SOD and Mn SOD):

Superoxide dismutases catalyze the reaction of superoxide ions and two protons to form hydrogen peroxide and O₂ Copper/Zinc SOD is found only in the cytosol of animal cells while homologues of this enzyme are found in both the cytosol and chloroplast in plants and algae. See Fridovich (1995), “Superoxide Radical and Superoxide Dismutases,” Annu. Rev. Biochem. 64:97-112. Manganese SOD is localized only to the mitochondria in any eukaryotic cell; Halliwell et al. (1999), “Antioxidant Defense Mechanism: From the Beginning to the End (of the Beginning),” Free Radic. Res. 31:261-272. Superoxide dismutases accumulate in response to stress, and are one of the main anti-oxidant defenses. Increased SOD levels have been linked to increased longevity, and increased tolerance to ischemic/reperfusion events and to factors that induce oxidative stress. High SOD levels indicate that the cell is responding to an oxidative stress; Mn SOD specifically shows that the mitochondria are experiencing an oxidative stress.

Cytochrome P450:

The isoforms of cytochrome P450 represent a family of enzymes involved with the metabolism of xenobiotics. Cytochrome P450 enzymes play a significant role in detoxifying the cell from a variety of insults including hydrocarbons. Each isoform is associated with a specific function and can therefore serve as a basis to distinguish between a variety of stressors. For example, cytochrome P450 1A1 hyperaccumulates in response to a benze(a)pyrene exposure. In contrast, cytochrome P450 3B1 does not hyperaccumulate in response to benze(a)pyrene, but does accumulate in response to high sterol or flavone exposure.

Cytochrome P450 2E is known to specifically oxidize ethanol to acetaldehyde via a monooxygenase mechanism, as well as other xenobiotics such as imidazole-based derivatives (Lieber (1997), “Cytochrome P-450 2E1: Its Physiological and Pathological Role,” Physiol. Rev. 77:517-544). Cytochrome P450 2E has both physiologically relevant oxidative and reductive reactions and is known to associate and catalyze as many as 60 xenobiotic-based substrates (Koop (1992), “Oxidative and Reductive Metabolism by Cytochrome P450 2E1,” FASEB J. 6:724-730; Lieber (1997, supra). For example, it causes the demethylation of N, N-dimethylnitrosamine and the hydroxylation of p-nitrophenol and chloroxazone (ibid.). Reduction reactions include reduction of a number of different lipid types. One of the primary reasons for using the 2E class of cytochrome P450s is that it is not induced by heat stress, but can respond to hypoxia/reperfusion events in mammals (Bayanov & Brunt, 1999). Cytochrome P450 2E is particularly useful as an indicator of a xenobiotic response of snails.

Table 1 describes the biomarkers that can be employed for species belonging to a particular group.

TABLE 1

| | Group of Species | | | | | |
|---|---|---|---|---|---|---|
| Biomarker name | Plant | Coral | Dinoflagellate | Arthropod | Mollusc | Fish |
| Mitochondrial Hsp70s | X | X | X | X | X | X |
| Mitochondrial Hsp60s | X | X | X | X | X | X |
| Chloroplast Hsp60 | X | | | | | |
| Chloroplast stromal | X | | | | | |
| Hsp70 | | | | | | |
| Mitochondrial sHsp | X | X | X | X | X | X |
| Chloroplast sHsp | X | | X | | | |
| Hsp90 (centrosome | X | X | X | X | X | X |
| homologue) | | | | | | |
| Mitochondrial SODs | X | X | X | X | X | X |
| Cytosolic SODs | X | X | X | X | X | X |
| Chloroplast SOD | X | | | | | |
| P-glycoprotein | | | | X | X | X |
| sHsp30 | | | | | | X |
| Mitochondrial dehydrin | X | | | | | |
| Chloroplast dehydrin | X | | | | | |
| GSH-peroxidase | X | | | X | X | X |
| GSH-transferase | X | | | X | X | X |
| Metallothionein I | | X | | X | X | X |
| Metallothionein II | | X | | X | X | X |
| Metallothionein III | | X | | X | X | X |
| Cytochrome P450 1A | | | | X | X | X |
| Cytochrome P450 2E | | | | X | X | X |
| Cytochrome P450 3A | | | | X | X | X |
| sHsp 26 | | X | | X | X | |
| Carbonyl (protein | X | X | X | X | X | X |
| adduct) | | | | | | |
| Malondialdehyde (lipid break | X | X | X | X | X | X |
| down product) | | | | | | |
| 8-oxo-deoxyguanosine | X | X | X | X | X | X |
| (DNA adduct) | | | | | | |
| PAH-protein (DNA | X | X | X | X | X | X |
| adduct) | | | | | | |
| Ubiquitin | X | X | X | X | X | X |

Abbreviations:
Hsp = heat-shock protein,
sHsp = small heat shock protein,
SOD = superoxide dismutase,
GSH glutathione,
PAH = polyaromatic hydrocarbon.

Once suitable biomarkers and species have been identified, an organism from the ecosystem of interest is obtained and tested. Broadly, the specific molecular biomarkers are measured in the species and compared against previously determined values established for normal, i.e., healthy, and abnormal, i.e., physiologically stressed, individuals. In this way, it is possible not only to determine the health of the organism in the particular ecosystem, but to determine the health of the ecosystem itself. Furthermore, if the levels of certain biomarkers are abnormal, it is possible to determine the prognosis for the ecosystem depending on, for example, the degree to which the measured values fall outside of normal.

Measuring the biomarker levels is conducted using any art known method. It is preferred, however, that an assay be used that is based on antigen-antibody interactions. Examples of such assays include agglutination assays, immunoprecipitation assays, and solid phase immunoassays. Particularly preferred solid phase immunoassays include enzyme immunoassays (EIA), e.g., ELISA, ELIPSOT or Western blot assays. Thus, the antigen, i.e., molecular biomarker, level can be effectively measured by employing assays that use antigen-antibody binding.

In developing antigen-antibody binding assays, care must be employed in determining the appropriate antigenic epitope or epitopes against which the antibody will be raised. Comparing the known amino acid sequence (obtained from Genbank or a literature search or derived experimentally using art-known techniques such as a protein sequencer) of the desired molecular biomarker to related proteins in other organisms allows for the design and/or selection of an antigen that is specific for the desired molecular biomarker in the taxonomical clade of interest. For example, mitochondrial-inducible Hsp70 has high homology between gastropods and bivalves. Thus, an antigen is designed such that the domain is conserved from gastropod species to bivalve species, but has no homology to domains in other Hsp70 isoforms. Generally, the antigen is about 15-25 amino acid residues in length, but larger and smaller antigens are contemplated as well.

Antibodies specific for the antigen (i.e., the molecular biomarker) must be developed for assays such as an enzyme immunoassay, e.g., ELISA, ELISPOT or Western blot. Any art-known technique may be employed to develop antibodies against the antigen of interest. Antibodies are preferably raised by introducing the antigen into a suitable animal, waiting a suitable time for antibodies to develop, and collecting and purifying the antibodies developed by the animal.

By way of a representative example, antibodies can be raised by injecting (e.g., via subcutaneous, intramuscular, intradermal, intravenous or intraperitoneal injection) into an animal an antigen optionally coupled to an immunogenic moiety. The immunogenic moiety may be, for example, KLH (keyhole limpet hemocyanin), diphtheria toxoid or bovine serum albumen, with KLH preferred. The immunogenic moiety is coupled to the antigen using conventional coupling reagents such as glutaraldehyde, m-maleimidobenzoyl-N-hydroxysuccinimide ester, carbodiimide or bis-biazotized benzidine. The use of the immunogenic moiety increases the yield of the antibody that is ultimately collected from the animal. Although any animal may be employed in raising the antibodies, a frog, mouse, rabbit, rat, or sheep is preferred. Rabbits, however, are particularly preferred animals for developing antibodies for use with the present method.

The desired antigens that are injected into the animal may be synthetically produced. Synthetic production of antigens generally employs techniques of standard solid phase peptide synthesis well known in the art. Briefly, the synthesis of peptides is sequentially carried out by incorporating the desired amino acid residues one at a time onto a growing peptide chain according to the general principles of solid phase synthesis as described, for example, by Merrifield (1963) J. Amer. Chem. Soc. 85:2149-2154. As is appreciated by those skilled in the art, the chemical syntheses of peptides generally requires the protection of reactive side chain groups of the various amino acid moieties with suitable protecting groups that prevent a chemical reaction from occurring at that site until the protecting group is ultimately removed. It will also be appreciated that is it necessary to protect the -amino group on an amino acid while that entity reacts at the carboxyl group, followed by the selective removal of the -amino protecting group to allow a subsequent reaction to take place at that site. Examples of suitable amino and side chain protecting groups are well known in the art.

Alternatively, the antigens can be obtained from an organism itself. For example, samples from the organism may be collected and the proteins containing the desired antigen recovered using conventional techniques such as, for example, centrifugation or size-exclusion chromatography.

Because of the relatively small size of antigens (generally about 15-25 amino acids in length), the epitope(s) associated therewith may be bound by only two to four immunoglobulins (e.g., IgGs). Thus, the developing antibodies may be described as “monospecific polyclonal” in nature. Consequently, this attribute results in a two- to four-fold increase in sensitivity over the corresponding monoclonal antibodies, in addition to an increase in the amount of antibodies produced.

Optimally, the animal is fed a special diet and quarantined to ensure that no antibodies are produced to extraneous antigens. In particular, it is critical that the animal does not develop antibodies against a different protein that will be tested.

Periodically, the antibody:antigen titer in the animal is determined. The animal may receive a booster injection of the antigen to increase the antibody yield if the titer is low. Alternatively, if a sufficient titer is found, the blood is collected via art-known techniques such as bleeding through an ear vein or employing a cardiac puncture.

Serum from the collected blood is separated via any art-known method such as centrifugation. Antibodies contained in the serum are then purified. A preferred method for purifying antibodies comprises employing a column, e.g., a HPLC (high pressure liquid chromatography), Protein A column. Protein A is a receptor capable of binding the Fc portion of antibodies. Thus, a sample containing antibodies is passed through a column containing immobilized Protein A. Once the sample has passed through the column, the bound antibodies are eluted by treatment with a suitable reagent, e.g., a strong acid or base. Thereafter, the serum containing the purified antibodies is mixed with a stabilizer solution so as to prevent degradation from freezing events. If desired, the serum can then be divided into aliquots and/or stored at −80 C.

Each set of purified antibodies is then incorporated into separate immunocolumns to create purification standards for each antibody. The antibodies are coupled to a solid phase substrate (available from Pierce Chemicals Co., Rockford Ill. or other commercial suppliers) using techniques known in the art or by following the instructions provided with the column. Samples from stressed organisms from each species are obtained and passed through each immunocolumn. Thereafter, bound proteins are eluted and collected. The eluants are first concentrated and then analyzed for purity and quality. Thereafter, the protein concentration is determined. As a further measure of validation, a portion from each of the eluants is fragmented by enzyme treatment, e.g., a trypsin treatment, and sequenced using standard techniques, e.g., Edman degradation sequencing.

The antibodies can be validated for specificity and cross-reactivity with the specific biomarker in the respective species and against the purified antigen. This is accomplished by using stressed specimens and running a 1D SDS-PAGE (1-dimensional sodium dodecylsulfate-polyacrylamide gel electrophoresis) and Western blotting.

The antibody titer is then determined for Western blotting and ELISA. Stressed specimens are used as validation samples to determine sample:antibody titer for ELISA analysis. In addition, protein concentration standards for each antibody is used to determine the range for each EIA for valid samples.

Once a suite of individual assays is created and validated and the ranges for normal and abnormal (i.e., nonstressed and stressed) levels of each physiological parameter are identified, organismal health can be assessed. Thus, a cellular or tissue sample is obtained from an organism located from the ecosystem of interest and homogenized. The homogenized liquid is then divided into aliquots and assayed to determine the level of each biomarker. For example, the homogenized liquid is placed in contact with an antibody that has been immobilized to a substrate. After a sufficient time has passed, the substrate is washed and a second antibody that has a detectable label is added, allowed to bind and subsequently washed. Suitable labels include, for example, chemiluminescent, colorimetric, enzymatic, fluorescent and radioactive moieties. For enzyme-linked antibodies, a substrate for the enzyme is added that provides a signal, e.g., color change. The signal is measured (e.g., via a desitometer) and the level of the biomarker calculated.

As will be appreciated, all antibody-antibody binding must be performed under suitable binding conditions. Typically, the temperature is from about 28 C to about 42 C, preferably from about 30 C to about 38 C, and most preferably at about 37 C. In addition, the assay is performed at a suitable pH, e.g., about 6.5 to about 8.5, preferably at pH 7.4. Furthermore, antigen-antibody complex formation is allowed to proceed for a time sufficient to substantially bind all antibodies to complementary antigens. It is preferred, however, that antigen-antibody complex formation is allowed to proceed for about 10 minutes to about 10 hours after the addition of the antigen.

Once the biomarkers are identified and quantitative assays for the biomarkers developed, samples are obtained from an appropriate organism in the ecosystem. Each assayed biomarker in the sample is measured and statistically analyzed against the “normal” or “nominal” value for that biomarker. The “normal” or “nominal” values for each biomarker can be determined from both laboratory and field studies. Alternatively, “normal” or “nominal” values may be obtained from the literature. In either case, the measured values of the biomarker data obtained from the organism are statistically compared to each of the corresponding “normal” or “nominal” values of each biomarker.

The statistical comparison can proceed in any method that provides a scientifically and statistically valid comparison of the biomarker values obtained from the organism to the corresponding normal values of each biomarker. A preferred method for statistical comparison of the biomarker data is known as analysis of variance (ANOVA). In operation, ANOVA tests whether several populations have the same mean by comparing how far apart the sample means are with how much variation there is within the samples. The ANOVA analysis includes an F statistic for testing the equality of several means and is expressed in terms of the F ratio: F=(variation among the sample means)/(variation among individuals in the same sample). If all of the sample means are identical, the F value is zero. In contrast, the F value gets larger when all the sample means are farther apart. Thus, large F values are evidence against the null hypothesis that all population means are the same. As a consequence, large F values indicate a significant deviation from normal for that biomarker. Such a deviation provides evidence (although not necessarily conclusive evidence) that the organism is experiencing the stress for which that particular biomarker traces.

In order to determine conclusively whether the organism is healthy or physiologically stressed, it is necessary to analyze at least two, and preferably more, biomarkers. Two and often more biomarkers are necessary to conclusively determine the health or stress of an organism because a deviation of a single physiological parameter may not necessarily cause an organism to be stressed. Thus, for example, an increase in a single cytochrome P450 isoform may indicate the presence of a xenobiotic (e.g., chemical contamination) in the organism, but the organism may have sufficiently compensated to maintain overall organismal health. A statistical increase in many cytochrome P450 isoforms, however, may indicate an assault of several xenobiotics or may indicate an organism that has been exposed to an overwhelming amount of the particular xenobiotic. In either case, the increase in three cytochrome P450 isomforms indicates a physiologically stressed organism. Analysis of several biomarkers can proceed by performing an ANOVA for each biomarker. Alternatively, a single multivariate analysis of variance (MANOVA) may be employed to analyze a series of biomarkers.

When the organism is determined to be healthy, the ecosystem from which that organism originated is also healthy, e.g., not deteriorating in either biodiversity or biomass. For example, an organism showing healthy levels of cytochrome P450 isoforms indicates that the ecosystem from which that organism originated is free from the xenobiotic(s) for which that biomarker traces. Performing other biomarker tests that measure other stressors provides additional information concerning the health of the ecosystem.

When the organism is determined to be unhealthy or physiologically stressed, the levels of each of the biomarkers are used to determine the type and degree of stress occurring in the organism and, by extenstion, in the ecosystem. Again, increases in the cytochrome P450 isoforms are indicative of xenobiotics (e.g., chemical contaminants) in the ecosystem. For certain organisms, e.g., shrimp, increases in Hsp70 , ubiquitin, carbonyl values and endosulfan/cysteine adduct (biomarkers that measure oxidative stress and protein degradation) are indicative of physiological stress from the presence of endosulfan in the ecosystem of interest. The degree of stress in the ecosystem is correlated with the degree of stress in the organism: the higher the difference between the measured value and the normal range for any given set of biomarkers, the higher the degree of stress in both the organism and ecosystem.

The method also determines whether the organism has adapted in response to the stress occurring in the ecosystem. Adaptation to a particular stressor can be determined by, for example, periodically repeating the assay on an organism from the same stressed population and comparing the measured results to previously obtained data. Stable biomarker values or values that progressively return closer to normal are indicative of an organism (and an ecosystem) that has adapted to the stress.

In addition, the method provides the ability to assess the physiological impact of the stress on the organism. For example, increases in Hsp70, ubiquitin, carbonyl values and endosulfan/cysteine adduct in shrimp indicates that endosulfan is forming a disulfide covalent bond with cysteine residues of proteins, including enzymes. Thus, the activity of these affected enzymes is inhibited. Other biomarkers are employed to provide similar assessments concerning the impact of other physiological stressors.

Prognostic evaluations of the ecosystem are also contemplated. Once the physiological stress has been identified, the biomarkers that trace the stressor are statistically analyzed to predict the probability of survival for that population which, in turn, is predictive for the ecosystem. For example, the appropriate biomarker data (e.g., Hsp70, ubiquitin, carbonyl values and endosulfan/cysteine adduct in shrimp stressed with endosulfan) can be analyzed using nominal logistic regression analysis to predict the probability of survival or estimate reproductive fitness. Alternatively, an above-normal (high) level of LPO and other biomarkers that trace oxidative stress in coral suggest that bleaching may occur within the next 2-3 months in the coral in that particular ecosystem.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All publications mentioned herein, both supra and infra, are hereby incorporated by reference. All ranges identified herein, both supra and infra, are inclusive.

Also, in these examples and throughout this specification, the abbreviations employed have their generally accepted meanings, as follows:

PVDF=polyvinylidenefluoride

SDS=sodium dodecylsulfate

EDTA=ethylenediamine tetraacetic acid

TBS=Tris-Buffered Saline

NADH=nicotinamide adenine dinucleotide

### EXPERIMENTAL:

### EXAMPLE 1

### Using Multiple Biomarkers to Assess the Health of Coral During Heat Stress

Coral reefs constitute some of the largest ecological structures on earth, and result from interactions between symbiotic organisms composed of a dinoflagellate algae (zooxanthellae) and scleractinian corals. Over the past two decades, and especially in the 1990's, coral reefs experienced extensive degradation worldwide. One etiology for this global degradation is a syndrome known as coral “bleaching” (Dustan (1999), “Coral Reefs under Stress: Sources of Mortality in the Florida Keys,” Nat. Res. Forum 23:147-155). Bleaching is a process whereby corals and other invertebrates harboring symbiotic algae lose their algae and/or experience degradation of the symbionts' photosynthetic pigments. In corals, loss of zooxanthellae can occur through expulsion via exocytosis, in situ digestion or by host cell detachment. Bleaching of corals has been linked to heat stress, cold stress, elevated visible light, light depravation, ultraviolet light exposure, low salinity, starvation and bacterial infection. However, evidence is increasing that heat stress is a primary factor in many, if not most, large-scale bleaching events (Brown, B. E. (1997), “Coral Bleaching: Causes and Consequences,” Coral Reefs 16, Suppl.:S 129-S 138).

In the 1980s, in situ observations and U.S. National Oceanic and Atmospheric Administration reports concerning Caribbean, Indian Ocean and South Pacific coral reefs suggested a relationship between pervasive coral bleaching and unusually warm periods (see, e.g., Brown et al. (1996), “Coral Bleaching Relative to Elevated Seawater Temperature in the Andaman Sea (Indian Ocean) over the Last 50 Years,” Coral Reefs 15:151-152). Recently, Stone and co-workers established a correlation between episodes of massive coral bleaching and increased severity and frequency of El Niño events (Stone et al. (1999), “Mass Coral Reef Bleaching: a Recent Outcome of Increased El Niño Activity?” Ecol. Letters 2:325-330). Increased ocean temperatures, compounded with other agents of coral degradation (e.g., pollution, disease, predation, etc.) pose an increasing and significant threat to the health and vitality of coral reef ecosystems worldwide.

Recent evidence indicates that heat stress induces concomitant oxidative stress and, in turn, may elicit the development of bleaching that can be alleviated with the exogenous addition of anti-oxidants (Lesser et al. (1994), “Effects of Morphology and Water Motion on Carbon Delivery and Productivity in the Reef Coral, Pocillopora damicornis (Linnaeus): Diffusion Barriers, Inorganic Carbon Limitation, and Biochemical Plasticity,” J. Exp. Mar. Biol. Ecol. 178:153-179; Lesser (1996), “Elevated Temperatures and Ultraviolet Radiation Cause Oxidative Stress and Inhibit Photosynthesis in Symbiotic Dinoflagellates,” Limnol. Oceanogr. 41:271-283; Lesser (1997), “Oxidative Stress Causes Coral Bleaching During Exposure to Elevated Temperatures,” Coral Reefs 16:187-192). Evidence also suggests that during heat stress the primary site of active oxygen generation is Photosystem II, an essential enzyme in photosynthesis found only in the chloroplasts of the zooxanthellae (Havaux (1992), “Stress Tolerance of Photosystem II In Vivo. Antagonistic Effects of Water, Heat, and Photoinhibition Stresses,” Plant Physiol. 100:424-432; Lesser (1996), “Elevated Temperatures and Ultraviolet Radiation Cause Oxidative Stress and Inhibit Photosynthesis in Symbiotic Dinoflagellates,” Limnol. Oceanogr. 41:271-283; Lesser (1997), “Oxidative Stress Causes Coral Bleaching During Exposure to Elevated Temperatures,” Coral Reefs 16:187-192; Warner et al. (1996), “The Effects of Elevated Temperature on the Photosynthetic Efficiency of Zooxanthellae In Hospite From Four Different Species of Reef Coral: A Novel Approach,” Plant Cell Environ. 19:292-299; Warner et al. (1999), “Damage to Photosystem II in Symbiotic Dinoflagellates: A Determinant of Coral Bleaching,” Proc. Natl. Acad. Sci. 96:8007-8012). Variation in coral bleaching may result from a complex interaction between active oxygen species generated by the zooxanthellae and the anti-oxidant ability of both the zooxanthellae and anthozoan (Dykens et al. (1982), “Oxygen Production by Endosymbiotic Algae Controls Superoxide Dismutase Activity in Their Animal Host,” Nature 297:579-580; Dykens et al. (1992), “Oxygen Radical Production in the Sea Anemone Anthopleura Elegantissima and Its Endosymbiotic Algae,” J. Exp. Biol. 168:219-241). Bleaching thus would allow the coral to deal with oxidative stress when other anti-oxidant defenses are overwhelmed. The method of the invention was employed in this example to assay specific parameters of the coral cell that are indicative of a non-stressed or stressed condition. The parameters evaluated included lipid peroxide levels (LPO), total glutathione (GSH), heat-shock protein 60 (Hsp60), heat shock protein 70 (Hsp70), B-crystallin homologue, chloroplast small heat-shock protein (chlpsHsp), homologues to the plant class I-IV small heat-shock proteins (sHsps), Mn superoxide dismutase (Mn SOD), Cu/Zn superoxide dismutase (Cu/Zn SOD), and ubiquitin. These parameters were chosen because they monitor specific cellular physiological functions. Measuring these parameters (1) indicates whether the structural integrity of the cell is challenged, (2) indicates whether there is a response to oxidative stress, (3) indicates whether metabolic processes (photosynthetic and oxidative phosphorylation) are or were subject to insult, and (4) provides evidence for identifying the stressor (i.e., heat, light, or both).

Materials and Methods:

All chemicals were obtained from Sigma-Aldrich (St. Louis, Mo., U.S.A.). PVDF and nitrocellulose membranes were obtained from Millipore Corp. (Bedford, Mass., U.S.A.). Dot blot and gel electrophoresis equipment were obtained from Bio-Rad Corp. (San Diego, Calif., U.S.A.). GSH-420 glutathione assay kits (Cat. #21023), LPO-560 assay kits (Cat. #21025), and antibody against Mn superoxide dismutase (Cat. #24327) were obtained from Oxis International, Inc. (Portland, Oreg., U.S.A.). Antibodies against Hsp70 (Cat. #SPA822), Hsp60 (Cat. #SPA805), B-crystallin (Cat. #SPA224), ubiquitin (Cat. #SPA200), and Cu/Zn superoxide dismutase (Cat. #SOD100) were obtained from StressGen Biotechnologies, Inc. (Victoria, British Columbia, Canada). Anti-rabbit and anti-mouse conjugated alkaline phosphatase antibodies were obtained from Promega (Madison, Wis., U.S.A.). Antibodies to the chloroplast small heat-shock protein and to Class I-IV plant small heat-shock proteins were produced as described in Heckathorn et al. (1998), supra, and Downs et al. (1998) Amer. J Bot. 85:175-183. Protein standards of Hsp70, Hsp60, and B-crystallin were obtained from StressGen Biotechnologies, Inc. Protein standards of ubiquitin, Cu/Zn SOD, and Mn SOD were obtained from Sigma-Aldrich. Protein standards for the chlpshsp and plant sHsp were produced as described in Heckathorn et al. (1998), supra, and Downs et al. (1998) FEBS Letters 430:246-250.

Coral Collection, Maintenance and Heat-Shock Conditions:

Skirt fragments from three colonies of Montastraea faveolata were collected near the Looe Key Special Use Zone south of Big Pine Key, Fla. (24° 34.142′ N, 81° 22.911 ′ W; depth -6 m). The fragments were transported to the Pigeon Key Marine Research Center and placed into closed 570 L aquaria. The following day, the fragments were cut into 5×5 cm squares using a diamond saw. The bases and sides were sealed with epoxy putty (Z-Spar Splash Zone epoxy). The coral explants were maintained in the aquaria at 27° C. under an irradiance of 350 moles photons m⁻² s⁻¹ (12 hr light: 12 hour dark), and were fed Artemia nauplii once per week. Control samples (three explants from each parent colony) were removed from these conditions and frozen.

Heat-shock experiments were conducted in the light and the dark. In both cases, the temperature was ramped from 27° C. to 33° C. over a three hour period and maintained at 33° C. for another nine hours. Heat-stress in the light was conducted from 08:00 until 20:00 and the dark experiment from 20:00 until 08:00 the following day. As with the controls, three explants from each parent colony (nine total) were used in each experiment, and all samples were frozen at the end of the experimental periods.

Coral Sample Preparation:

Samples of frozen coral tissue were scraped with a spatula and suspended in a solution consisting of 10 mM phosphate buffer (pH 7.8), 5 mM butylhydrotoluene, and 0.5% SDS. Samples on ice were sonicated until coral tissue was homogenized. Samples were then centrifuged and supernatant placed in a new tube and stored at −80° C. until sample analysis for LPO and GSH. Protein concentration of samples were assayed by the method of Ghosh et al. (1988), “Use of a Scanning Densitometer or an ELISA Plate Reader for Measurement of Nanogram Amounts of Protein in Crude Extracts From Biological Tissue,” Anal. Biochem. 169:227-233.

After performing LPO, GSH, and protein concentration assays, 100 L of a solution containing 20% SDS, 50 mM Tris-HCl (pH 7.8), 100 mM dithiothreitol, 80 mM EDTA, 3% polyvinyl pyrrolidone (w/v), 20 mM phenylmethylsulfonyl fluoride, 20 mM benzamide, 50 M -amino-caproic acid, and 1 g pepstatin A was added to 900 L of sample. Samples were then boiled for three minutes, allowed to sit at 25° C. for five minutes and then centrifuged at 10,000× g for five minutes. Supernatant was transferred to a new tube and the pellet was discarded. Samples were then subjected to another protein concentration assay.

Spectrophotometric Assays:

Samples were analyzed for LPO and total GSH content following the manufacturer's instructions. Each assay was done in triplicate.

Dot Blot, Gel Electrophoresis, Immunoblotting, and Densitometric Analysis:

Samples were then assayed for Hsp70, Hsp60, B-crystallin, chlpsHsp, sHsps, ubiquitin, Cu/Zn superoxide dismutase, and Mn superoxide dismutase using immunochemical analysis. All samples were done in triplicate. ELISPOT technique on nitrocellulose membrane was employed by using a dot blot apparatus. Western blotting on PVDF membrane was used after samples were subjected to SDS-PAGE. Both types of blots were then blocked for one hour in either 5% non-fat-dried milk in 1×TBS or for ubiquitin blots, blocked for 1 hour in 0.1% Tween-20 solution. Blocking solution was decanted and blots were incubated in the appropriate primary antibody solution for 12 hours at 4° C. Primary antibody solution was decanted, blots subjected to four 10-minute washes in 1×TBS, and then incubated in the appropriate secondary antibody solution for one hour. Secondary antibody solution was decanted, blots were again subjected to four 10-minute washes in lx TBS, then developed in a nitroblue tetrazolium/5-bromo-4-chloro-3-indolyl phosphate solution.

Once developed, blots were scanned into a computer and analyzed using NIH image software (National Institutes of Health, Bethesda, Md., U.S.A.). Concentration standards for each assay were determined and a quadratic or polynomial equation was used to determine the concentration of each sample.

Cellular Parameters:

A suite of molecular and cellular biomarkers was used to detect physiological changes at a subcellular level in response to light and heat stress. The biomarkers included general indicators of cell integrity (LPO, GSH, and ubiquitin), molecular chaperones that indicate the integrity of enzyme pathways (Hsp 60 and Hsp 70), small heat shock proteins produced in response to heat stress (B-crystallin, chlpsHsp, and plant sHsp), and anti-oxidant enzymes indicative of oxidative distress (Cu/Zn SOD and Mn SOD).

Statistical Analyses:

Mixed model analysis of variance (ANOVA) was used with treatments as fixed effects and coral clones as random effects to test the null hypotheses that mean biomarker levels did not vary among treatments or coral clones. When preliminary analyses showed that clone effects were small (F-statistics near 1.00), their sums of squares were pooled with the residual error to maximize statistical power, as recommended by Sokal and Rohlf (1995) Biometry (W. H. Freeman, N.Y.). Separate ANOVAs were performed on each bioassay. The Dunn-Sidák method (ibid.) was used and a critical probability of P′=0.00512 was set for each test to maintain the experiment-wise Type I error rate at =0.05 for all ten ANOVAs combined. When ANOVA identified significant differences among treatment means, the Tukey-Kramer Honestly Significant Difference (HSD) method was used as an exact alpha-level test for all differences between means (ibid.). These conservative procedures limited the probability of rejecting a true null hypothesis to the desired (=0.05) level. All statistical analyses were performed using JMP v. 3.2.2 (SAS Institute, Inc., Cary, N.C., U.S.A.).

Results:

For immunochemical assays of cellular parameters, coral samples were prepared and subjected to SDS-PAGE with an appropriate standard (e.g., purified Hsp70 was run along-side coral sample and assayed with Hsp70 antibody), to insure that the antibody cross-reacted with an appropriate homologous protein of appropriate size and did not demonstrate significant non-specific cross-reactivity with other proteins (quality control)(data not shown). At least five different antibodies for a specific parameter were sampled for quality control.

Treatments had a significant effect on mean levels of all biomarkers except Hsp60 (F_{2,2}=18.96, P=0.0091; Table 2). However, treatments explained substantial amounts of variation in this biomarker (R²=0.68). Coral clones only had a significant effect on mean GSH levels (F_{2,2}=30.10, P=0.004). Treatment means, their standard errors, and test statistics are reported in Table 2.

TABLE 2
Heat Stress

| Biomarker | Control | HS-dark | HS-light | F |
|---|---|---|---|---|
| GSH | 2.57^{a} ± 0.186 | 1.36^{b} ± 0.145 | 1.30^{b} ± 0.252 | 137.2** |
| LPO | 12.7^{a} ± 0.52 | 56.0^{b} ± 4.46 | 75.4^{c} ± 4.39 | 132.8** |
| ubiquitin | 132^{a} ± 3.7 | 437^{b} ± 10.9 | 619^{c} ± 11.4 | 2191.8*** |
| Hsp60 | 23.7 ± 2.03 | 46.7 ± 6.49 | 43.3 ± 5.17 | 18.9ns |
| Hsp70 | 337^{a} ± 6.7 | 681^{b} ± 30.1 | 688^{b} ± 26.6 | 103.6** |
| B- | 28.0^{a} ± 3.06 | 63.7^{b} ± 1.86 | 57.0^{b} ± 1.73 | 68.4*** |
| crystallin | | | | |
| chlpsHsp | 10.0^{a} ± 3.61 | 13.0^{a} ± 2.65 | 58.0^{b} ± 4.36 | 216.9*** |
| plant sHsp | 16.7^{a} ± 4.48 | 34.7^{b} ± 2.60 | 53.7^{c} ± 4.91 | 102.7** |
| Cu/Zn | 30.7^{a} ± 3.93 | 53.0^{b} ± 2.08 | 69.3^{c} ± 1.67 | 50.1** |
| SOD | | | | |
| MnSOD | 21.7^{a} ± 2.40 | 35.7^{b} ± 1.76 | 40.3^{b} ± 0.33 | 31.5** |
| In Table 2, which provides a summary of the biomarker responses to experimental treatments in star coral (Montastraea faveolata), treatments were optimal laboratory conditions (control), high-temperature stress in the absence of light for 10 hours (HS-dark), and high-temperature stress under nominal photosynthetic active radiation (PAR) for 10 hours (HS-light). Entries in the table give treatment means ± 1 SE, F-statistics, and | | | | |
| #significance levels; | | | | |

ns = not statistically significant,
*P < 0.005,
**P < 0.001,
***P < 0.0001.
Treatment means with different superscripted letters differed significantly at = 0.05 using the Tukey-Kramer HSD Method. Units are: M GSH; g total protein; M LPO/mg total protein; nanog ubiquitin/ g total protein; pg Hsp or B-crystallin/ g total protein; and relative protein concentrations for SODs.

Biomarkers for Anthozoan Responses:

Three biomarkers measured only the anthozoan's physiological responses to stress: B-crystallin, Cu/Zn SOD, and Mn SOD. Levels of both B-crystallin and Mn SOD were significantly higher in both heat-stress treatments than in the nonheat-stressed control. Levels of B-crystallin and Mn SOD were not significantly different from samples heat-stressed in the dark and samples heat-stressed in the light (Table 2). Levels of B-crystallin more than doubled in heat-stressed treatments compared to controls. Manganese SOD, which is produced when anthozoan mitochondria are oxidatively stressed, increased 75% in heat-stressed treatments compared to controls. In contrast, Cu/Zn SOD, which indicates greater oxidative stress in the cytosol, differed significantly among all three treatments. Its levels were 1.7 and 2.3 times greater when anthozoans were heat-stressed in the dark and light, respectively, than in controls (Table 2).

Biomarkers for Zooxanthellae Responses:

Two biomarkers measured only the zooxanthellae's physiological responses to stress: chlpsHsp and plant sHsp. Levels of chlpshsp were significantly higher in corals exposed to both heat-stress and PAR than to either control or heat-stress and no light conditions. This is analogous to the behavior of the chlpsHsp homologue in vascular plants (Downs et al. (1999), “The Chloroplast Small Heat-Shock Protein: Evidence for a General Role in Protecting Photosystem II Against Oxidative Stress and Photoinhibition,” J. Plant. Physiol. 55:488-496.). In the coral's zooxanthellae, chlpsHsp levels increased more than five-fold (Table 2). Levels of plant shsp differed significantly among all three treatments, with production 2.1 and 3.2 times greater when coral were heat-stressed in the dark and light, respectively, than in controls (Table 2).

### EXAMPLE 2

### Using Multiple Biomarkers to Assess the Health of Grass Shrimp Exposed to Natural and Anthropogenic Stressors

A molecular biomarker system of the invention was used to assay specific parameters of the shrimp cell indicative of a non-stressed or stressed physiological condition. The cellular parameters included: lipid peroxide levels (LPO); total glutathione (GSH); heat-shock protein 60 (Hsp60); heat-shock protein 70 (Hsp70); B-crystallin homologue (sHsp); manganese superoxide dismutase (Mn SOD); cytochrome P450 2E1; metallothionein class I and II; and ubiquitin. As in Example 1, these parameters were chosen because they represent specific cellular physiological functions. The levels of these parameters were determined in Palaeomonetes pugio (grass shrimp) under control conditions and when exposed to five different stressors: (1) heat, (2) cadmium, (3) atrazine, (4) water-accommodating fraction of diesel fuel, and (5) water-accommodating fraction of bunker fuel #2. The example demonstrates that the method and system of the invention can differentiate between P. pugio that experienced these different stressors.

Materials and Methods:

All chemicals for buffered solutions and cadmium chloride were obtained from Sigma Chemical Co. (St. Louis, Mo., U.S.A.). Atrazine was obtained from Supelco Chemical (Bellefonte, Pa., U.S.A.). Diesel and bunker fuel #2 were generous gifts from Exxon (Charleston, S.C., USA). PVDF and nitrocellulose membranes were obtained from Millipore Corp. (Bedford, Mass., U.S.A.) Dot blot and gel electrophoresis equipment were obtained from Bio-Rad Corp. (San Diego, Calif., U.S.A.) GSH-420 glutathione assay kits (Cat. #21023) and LPO-560 assay kits (Cat.#21025) were obtained from Oxis International, Inc. (Portland, Oreg., U.S.A.). Antibodies against Hsp70 (Cat. #SPA822), Hsp60 (Cat. #SPA805), and B-crystallin (Cat. #SPA224), and ubiquitin (Cat. #SPA200) were obtained from Stressgen Biotechnologies, Inc. (Victoria, British Columbia, Canada). Metallothionien antibodies against MT-Class I (Cat.#18-0133) was obtained from Zymed (South San Francisco, Calif., U.S.A.). Anti-rabbit and anti-mouse conjugated alkaline phosphatase antibodies were obtained from Promega (Madison, Wis., U.S.A.) Protein standards of Hsp70, Hsp60, B-crystallin was obtained from Stressgen Biotechnologies. Antibody to Cytochrome P450 2E 1 and manganese superoxide dismutase (Mn SOD) were generous gifts from Oxis International. Protein standards of ubiquitin, Mn SOD, and metallothionein were obtained from Sigma.

Collection and Growth Condition of P. pugio:

Palaeomonetes pugio (grass shrimp) were collected by dip netting in the Wadmalah River near Charleston, S.C., U.S.A. This site is considered an unimpacted reference site and is a long-term ecological monitoring site for both the South Carolina Department of Natural Resources and U.S. National Oceanic and Atmospheric Administration Ecotoxicology Program. Water and sediment sample analyses are conducted at least on a yearly basis for a number of pesticides, herbicides, metal, polyaromatic hydrocarbons, and eutrophic parameters (US NOAA NOS ORCA 128; SCDNR & US NOAA NMFS Tidal Creek Project).

Grass shrimp were acclimated to the laboratory at least one month prior to stress challenges. Preliminary experiments were carried out to determine the optimal means of growing and handling shrimp to induce a minimal stress response during laboratory experiments. Shrimp were grown at 22-24° C. with a 14 hour photoperiod in 80 liter tanks filled with filtered, brackish water (20 ppt salinity), the sides of the tanks were covered with black, light-impermeabable cloth. Stress Exposures:

Transferring of shrimp from culture tanks to dosing chambers induced a significant response for many parameters, which is normal for many experimental organisms, and especially for grass shrimp (Oberdorster et al., 1999, citation provide infra). Background stress levels were according substantially reduced by acclimating the shrimp in their dosing chambers seven days before the stress challenge. One shrimp (25-35 mg wet weight) was placed in a 1 L glass dosing chamber filled with 400 mL of filtered, brackish water (20 ppt salinity). During the seven-day acclimation, grass shrimp were subject to a 25 mol m⁻²s⁻² photosynthetic-active-radiation 14 hour photoperiod at 22° C. 300 mL of the brackish water in the dosing chamber was changed with fresh brackish water on day 2, 4, and 6 and fed brine shrimp on the 1^{st}, 3^{rd}, and 5^{th} day of dosing chamber acclimation. Dissolved oxygen, ammonia content, and temperature were continually monitored throughout the seven-day chamber acclimation. Dosing chambers were labeled for the type and dose of the stress with five replicates per treatment. Dosing chambers were then arranged in a randomized block design for stress challenging (Sokal and Rohlf, 1995). At the end of each 8-hour challenge, shrimp were immediately collected and frozen in liquid nitrogen and kept at −80° C. until sample preparation.

Heat stress—Grass shrimp were subject to an 8-hour temperature exposure of either 22° C., 30° C., 38° C., and 41° C. In heat-stress treatments, the temperature was ramped from 22° C. to either 30° C., 38° C. or 41° C. over a 70-minute period.

Cadmium stress—Grass shrimp were subjected to an 8-hour exposure to 0 M, 1 M, 5 M, and 50 M cadmium chloride. The solvent carrier was water.

Atrazine stress—Grass shrimp were exposed to 1 g/L, 500 g/L, or 1 mg/L of an acetone stock of technical grade atrazine (99%). Control animals were exposed to 50 L of acetone/400 mL brackish water, which was equal to the highest volume of solvent solution delivered to atrazine-challenged samples.

Diesel fuel and Bunker fuel #2 stress—Grass shrimp were exposed to either 0 g/L, 0.5 g/L, 1 g/L, and 3 g/L water accommodating fraction (WAF) of diesel or bunker fuel, which was prepared according to Blenkisopp et al. (1996), “How to Prepare Water Accommodated Fractions From Petroleum Hydrocarbons For Use in Aquatic Toxicity Testing—The Basics,” Proc 19^{th} Arctic and Marine Oilspill Program Technical Seminar, Environmental Canada, Ottawa, Ontario, pp. 515-528. One liter of brackish water was added to Teflon-coated 1.1 L Nalgene bottles (Nalge Company, Rochester, N.Y., U.S.A.). The appropriate amount of diesel or bunker fuel was added to each bottle and allowed to mix, using a magnetic stirrer, for 48 hours. Brackish water was used as a control.

Sample Preparation:

Shrimp were frozen in liquid nitrogen, ground in liquid nitrogen to a fine powder using a mortar and pestle, and then suspended in a solution consisting of 10 mM phosphate buffer (pH 7.8), 5 mM butylhydrotoluene, and 0.5% SDS. Samples were vortexed for 30 seconds and then centrifuged at 10,000× g for 5 minutes. The supernatant was placed in a new tube for LPO and GSH analyses. Protein concentration of samples were assayed by the method of Ghosh et al. (1988), supra.

After performing LPO, GSH and protein concentration assays, 100 L of a solution containing 20% SDS, 50 mM Tris-HCl (pH 7.8), 100 mM dithiothreitol, 80 mM EDTA, 3% polyvinyl pyrrolidone (w/v), 20 mM phenylmethylsulfonyl fluoride, 20 mM benzamide, 50 M—amino-caproic acid, and 1 g pepstatin A was added to 900 L of sample. Samples were then boiled for 3 minutes, allowed to sit at 25° C. for 5 minutes, and then centrifuged at 10,000× g for 5 minutes. Supernatant was transferred to a new tube and the pellet was discarded. The sample was then subjected to another protein concentration assay.

Spectrophotometric Assays:

Samples were analyzed for LPO and total GSH content following the manufacturer's instruction. Samples for each assay were analyzed in triplicate, and sample means provided independent unit of statistical analysis.

Dot Blot, Gel Electrophoresis, Immunoblotting, and Densitometric Analysis:

Samples were then assayed for Hsp70, Hsp60, B-crystallin, ubiquitin, cytochrome P450 2E1, Mn SOD, and metallothionein using immunochemical analysis. All samples were analyzed in triplicate. ELISPOT technique on nitrocellulose membrane was employed using a dot blot apparatus. Western blotting on PVDF membrane was used after samples were subjected to SDS-PAGE. Both types of blots were blocked for 1 hour in either 5% non-fat-dried milk in 1×TBS (50 mM Tris/HCl (pH 9.8), 10 mM NaCl) or, for ubiquitin blots in 0.1% Tween-20 TBS solution. Blocking solution was decanted and blots were incubated in the appropriate primary antibody solution for 12 hours at 4° C. Primary antibody solution was decanted, blots subjected to four 10-minute washes in 1×TBS, and then incubated in the appropriate secondary antibody solution for 1 hour. Secondary antibody solution was decanted, blots were again subjected to four 10-minute washes in 1×TBS, then developed in a nitroblue tetrazolium/5-bromo-4-chloro-3-indolyl phosphate solution.

Once developed, blots were scanned into a computer and analyzed using NIH image software (Http://rsb.info.nih.gov/nih-image). A serial dilution of purified protein for each cellular parameter was included in each assay to allow sample quantification and assay quality control. For example, titered concentrations of purified metallothionein from rabbit were used as both a qualitative and quantitative standard for the samples assayed with metallothionein. Concentration standards for each assay were determined and a quadratic or polynomial equation was used to determine the concentration of each sample.

To determine that the antibodies bound to their specific targets, preliminary experiments were conducted to ensure the validity of the antibody assays. Shrimp were exposed either to high temperature, cadmium, ethanol, or acetone. Shrimp were then homogenized in an SDS/50 mM dithiothreitol buffered solution, subjected to SDS-PAGE, immunoblotted to PVDF membrane, and assayed with at least 3 different antibodies per cellular parameter. Antibodies finally chosen as the enzyme immunoassay (EIA) had the best signal to noise ratio for each cellular parameter. Purified protein of each cellular parameter was run along side control and treated samples.

Because of the uniqueness of each of the cellular parameters, specific modifications were made for each ELISPOT assay. For example, ELISPOT analyses of metallothionein proteins followed a slightly modified protocol described by Mizzen et al., (1996). Small heat-shock proteins or B-crystallin analysis employed higher concentrations of SDS and DTT in the buffer and were boiled for 3 minutes to prevent oligomerization of the sHsps under mild or non-denaturing conditions.

Statistical Analysis:

Multivariate analysis of variance (MANOVA) was used to simultaneously assess the responses of all nine biomarkers in each experiment. MANOVA provided a single test of the null hypothesis that stressor levels had no effect on the multidimensional vector of mean biomarker responses. Significance was assessed using Wilks' (Morrison (1976), Multivariate Statistical Methods, McGraw-Hill, New York, N.Y., U.S.A.). When MANOVA revealed significant differences among treatments, separate univariate analyses of variance (ANOVAs) were used to indicate which response variables contributed most strongly to these differences. The small size of the heat stress experiment and non-normally distributed residuals in the cadmium chloride experiment prevented this multivariate approach. Instead, the Dunn-Sidak method (Sokal and Rohlf, 1995) was used and set a critical probability of ′=0.00568 for each test, which maintained the experimentwise Type I error rate at =0.05 for all nine ANOVAs combined.

When ANOVA identified significant differences among treatment means for a particular biomarker, the Tukey-Kramer Honestly Significant Difference (HSD) method was used as an exact alpha-level test for all differences between means (Sokal and Rohlf, 1995). These conservative procedures limited the probability of rejecting a true null hypothesis to the desired experimentwise level (p=0.05). Logarithmic transformations of some data were required to meet the assumptions of MANOVA and ANOVA (independent, normal, and homoscedastic residuals: Morrison, 1976; Zar, 1999). These included Hsp60, Hsp70 and Mn SOD in the diesel fuel experiment, and Hsp60 in the atrazine experiment. However, for ease of interpretation all tables report untransformed treatment means and their standard errors.

Canonical correlation analysis (CCA) was as a heuristic tool to illustrate how biomarkers could be used to discriminate among environmental stressors. CCA is an eigen analysis method that reveals the basic relationships between two matrices (Gauch (1985), Multivariate Analysis in Community Ecology, Cambridge University Press, New York, N.Y. , U.S.A.), in this case those of stressor treatments and biomarker data. The CCA provided an objective statistical tool for determining which biomarker (or suite of biomarkers) best revealed the presence of a particular environmental stressor. This analysis required combining data from all six experiments into one matrix, which was done by expressing biomarker responses in a given treatment as a proportion of their mean level in the control. Two assumptions of CCA, that stressor gradients were independent and linear, were constraints of the experimental design. The other main assumption, that biomarker responses were linear, was met with few exceptions (GSH responses to diesel fuel and heat stress, and Hsp60 responses to atrazine). All statistical analyses were performed using JMP v. 3.2.2 (SAS Institute, Inc., Cary, N.C., U.S.A.).

Results:

Antibody validation: The metallothionein antibody cross-reacted with three cadmium-inducible proteins between ca. 7 k and 12 kDa, but was unable to detect these proteins in the controls or other treatments. Cytochrome P450 2E 1 cross-reacted with two ethanol- and acetone-inducible proteins of ca. 53 kDa. Both these proteins were detectable in controls and other treatments, but cadmium and heat stress did not elicit significant accumulation. Hsp60 antibodies cross-reacted with at least two protein bands of about 62 kDa and 66 kDa that were inducible in all treatments. The Hsp70 antibody cross-reacted with two to three proteins between ca. 70 kDa and 72 kDa. Hsp70 homologues were inducible by all treatments except ethanol and acetone (data not shown). B-crystallin antibody cross-reacted with a single, ca. 19-21 kDa protein that was inducible by all treatments but undetectable in the control samples. This antibody did not cross-react with the sHsp22 or sHsp26 (p26) protein homologues of Artemia nor Drosophila sHsp26 (data not shown). Mn SOD antibody cross-reacted with a single protein of about 23 kDa that was inducible by both heat stress and cadmium, but not by exposure to ethanol or acetone.

Stress Responses: Treatments had significant effects on the vector of biomarker responses in the heat stress, atrazine, bunker fuel and diesel fuel experiments (all Wilks'<0.026 and approximate F>2.25, all P<0.03; Table 3). Treatment means, their standard errors, and test statistics are reported in Tables 3-6.

TABLE 3
Heat Stress

| Biomarker | Control | Low | Medium | F |
|---|---|---|---|---|
| GSH | 1.91^{a} ± 0.607 | 10.6^{b} ± | 14.1^{b} ± 2.11 | 16.6*** |
| | | 1.521 | | |
| LPO | 0.71^{a} ± 0.013 | 0.86^{b} ± | 1.23^{b} ± | 28.4**** |
| | | 0.053 | 0.066 | |
| ubiquitin | 1.76^{a} ± 0.15 | 2.10^{b} ± | 2.36^{c} ± | 5.35* |
| | | 0.055 | 0.051 | |
| Hsp60 | 195.4^{a} ± 15.1 | 264^{a,b} ± 32.9 | 311.8^{b} ± 21.6 | 9.63** |
| Hsp70 | 16.1^{a} ± 1.80 | 21.58^{ab} ± 1.63 | 24.92^{b} ± 2.28 | 5.77* |
| B- | 403.4^{a} ± 56.2 | 886.8^{b} ± 26.8 | 1117.6^{b} ± 96.7 | 30.1**** |
| crystallin | | | | |
| Mn SOD | 3.32^{a} ± 0.28 | 9.89^{ab} ± 1.48 | 13.03^{b} ± 2.78 | 7.35*** |
| Metallo. | 3.478 ± 0.17 | 2.98 ± 0.31 | 3.68 ± 0.25 | 2.02ns |
| Cyt. P450 | 14.93 ± 1.90 | 17.1 ± 0.27 | 16.98 ± 0.72 | 1.06ns |

Table 3. Summary of biomarker responses to heat stress in grass shrimp (Palaeomonetes pugio). Treatments were 22° C. (control), 30° C. (low), and 38° C. (medium).Entries in the table give treatment means ± 1 SE, F-statistics, and significance levels;
ns = not statistically significant,
*P < 0.05,
**P < 0.01,
***P < 0.001,
***P < 0.0001.
Treatment means with different superscripted letters differed significantly at = 0.05 using the Tukey-Kramer HSD Method. Units: mM GSH/mg total protein; mM LPO/mg total protein; nanog ubiquitin/ g total protein; ng Hsp or B-crystallin/ g total protein; metallothionine are ng metallothionein/mg total protein; Mn SOD is g SOD/ mg total protein; relative protein concentrations cyt P450.

TABLE 4
Cadmium Chloride Stress

| Biomarker | Control | Low | Medium | High | F |
|---|---|---|---|---|---|
| GSH | 1.06^{a} ± 0.042 | 1.21^{a} ± 0.053 | 1.40^{a} ± 0.069 | 1.83^{b} ± 0.151 | 14.0*** |
| LPO | 3.9^{a} ± 0.65 | 8.5^{b} ± 0.85 | 13.2^{c} ± 0.73 | 17.2^{c} ± 1.84 | 26.5*** |
| ubiquitin | 2.20^{a} ± 0.228 | 4.36^{b} ± 0.121 | 4.74^{bc} ± 0.178 | 6.16^{c} ± 0.674 | 19.4*** |
| Hsp60 | 114^{a} ± 7.7 | 153^{a,b} ± 10.7 | 168^{b} ± 14.8 | 183^{b} ± 10.9 | 6.84* |
| Hsp70 | 22^{a} ± 0.6 | 30^{a} ± 0.9 | 27^{a} ± 0.9 | 47^{b} ± 5.3 | 24.3** |
| B-crystallin | 380^{a} ± 30 | 500^{ab} ± 43 | 660^{b} ± 46 | 930^{c} ± 68 | 23.8*** |
| SOD | 2.3 ± 0.07 | 3.3 ± 0.12 | 4.3 ± 0.80 | 4.0 ± 0.16 | 4.40ns |
| Metallo. | 2.8^{a} ± 0.57 | 13.6^{b} ± 0.79 | 15.0^{bc} ± 0.83 | 17.5^{c} ± 1.36 | 48.0*** |
| Cyt. P450 | 14.5 ± 1.25 | 14.5 ± 1.19 | 13.4 ± 1.48 | 12.9 ± 1.26 | 0.37ns |

Table 4. Summary of biomarker responses to cadmium chloride stress in grass shrimp (Palaeomonetes pugio). Treatments were 0 uM (control), 1 uM (low), 5 uM (medium), and 50 uM (high) of cadmium chloride. Entries in the table give treatment means ± 1 SE, F-statistics from one-way or Welch ANOVA, and significance levels;
ns = not statistically significant,
*P < 0.00568 (adjusted for multiple tests; see text),
**P < 0.001,
***P < 0.0001.
Units of measurement are as in Table 3.

TABLE 5a
Bunker fuel stress
MANOVA results: Wilks′ = 0.0026,
approx. F_{27,24} = 5.94, P < 0.0001.

| Biomarker | Control | Low | Medium | High | F |
|---|---|---|---|---|---|
| GSH | 1.4^{a} ± 0.26 | 3.3^{a} ± 0.54 | 3.8^{ab} ± 0.66 | 6.1^{b} ± 0.81 | 10.2*** |
| LPO | 0.68^{a} ± 0.128 | 1.56^{b} ± 0.051 | 1.64^{b} ± 0.087 | 1.58^{b} ± 0.097 | 23.3*** |
| ubiquitin | 1.22^{a} ± 13 | 1.68^{ab} ± 0.185 | 1.60^{ab} ± 0.071 | 1.80^{b} ± 0.138 | 3.87* |
| Hsp60 | 130^{a} ± 7.7 | 250^{a} ± 45 | 280^{a} ± 37 | 660^{b} ± 62 | 28.0*** |
| Hsp70 | 14 ± 1.5 | 18 ± 1.7 | 21 ± 3.2 | 21 ± 2.1 | 2.09ns |
| B-crystallin | 310 ± 14 | 220 ± 37 | 230 ± 40 | 190 ± 27 | 2.78ns |
| SOD | 1.4^{a} ± 0.30 | 4.4^{ab} ± 1.07 | 4.7^{ab} ± 0.56 | 6.3^{b} ± 1.33 | 4.91* |
| Metallo. | 1.16 ± 0.218 | 1.00 ± 0.202 | 1.34 ± 0.169 | 1.40 ± 0.195 | 0.85ns |
| Cyt. P450 | 14^{a} ± 1.0 | 16^{a} ± 1.3 | 26^{b} ± 1.8 | 25^{b} ± 3.5 | 8.08** |

Table 5a and 5b. Summary of biomarker responses to bunker and diesel fuel stress in grass shrimp (Palaeomonetes pugio). Treatments were 0 g/L (control), 0.5 g/L (low), 1 g/L (medium), and 3 g/L (high) of water-accommodating fraction of bunker or diesel fuel for tables 5a and 5b, respectively. Table entries and units of measurement are as in Table 3.

TABLE 5b
Diesel fuel stress

| Biomarker | Control | Low | Medium | High | F |
|---|---|---|---|---|---|
| GSH | 1.6^{a} ± 0.34 | 7.7^{bc} ± 1.49 | 4.5^{ab} ± 1.30 | 10.4^{c} ± 1.29 | 10.2** |
| LPO | 0.60^{a} ± 0.031 | 0.90^{a} ± 0.181 | 1.03^{a} ± 0.133 | 2.14^{b} ± 0.175 | 22.0*** |
| ubiquitin | 0.98^{a} ± 0.124 | 1.04^{a} ± 0.175 | 1.36^{ab} ± 0.060 | 1.56^{b} ± 0.024 | 5.96** |
| Hsp60 | 80 ± 16 | 80 ± 15 | 169 ± 84 | 130 ± 18 | 0.80ns |
| Hsp70 | 50^{a} ± 4 | 80^{ab} ± 15 | 164^{bc} ± 60 | 300^{c} ± 18 | 14.2*** |
| B-crystallin | 420^{a} ± 79 | 830^{b} ± 109 | 869^{b} ± 116 | 1410^{c} ± 79 | 17.5*** |
| SOD | 4.3^{a} ± 0.35 | 8.5^{ab} ± 1.93 | 11.8^{ab} ± 1.77 | 8.8^{b} ± 0.88 | 7.73** |
| Metallo. | 2.7 ± 0.34 | 2.9 ± 0.39 | 2.7 ± 0.41 | 2.9 ± 0.18 | 0.12ns |
| Cyt. P450 | 22^{a} ± 1.7 | 56^{b} ± 5.9 | 64^{bc} ± 9.1 | 85^{c} ± 7.9 | 15.2*** |

TABLE 6
Atrazine stress
MANOVA results: Wilks′ = 0.0251,
approx. F_{27,24} = 2.25, P < 0.025.

| Biomarker | Control | Low | Medium | High | F |
|---|---|---|---|---|---|
| GSH | 5.4^{a} ± 1.42 | 10.6^{a} ± 2.81 | 7.0^{a} ± 1.23 | 16.0^{b} ± 1.26 | 6.78* |
| LPO | 1.00^{a} ± 0.035 | 0.76^{b} ± 0.060 | 0.74^{b} ± 0.040 | 0.62^{b} ± 0.020 | 14.6*** |
| ubiquitin | 1.58 ± 0.132 | 1.44 ± 0.068 | 1.38 ± 0.066 | 1.48^{b} ± 0.058 | 0.95ns |
| Hsp60 | 130^{a} ± 20 | 80^{a} ± 16 | 260^{a} ± 108 | 640^{b} ± 29 | 10.38*** |
| Hsp70 | 6.9^{a} ± 0.72 | 9.2^{ab} ± 1.31 | 10.3^{ab} ± 2.16 | 14.0^{b} ± 1.69 | 3.59* |
| B-crystallin | 280 ± 22 | 290 ± 27 | 250 ± 45 | 190 ± 27 | 2.32ns |
| SOD | 2.7 ± 0.82 | 4.3 ± 0.23 | 4.6 ± 1.61 | 7.9 ± 1.86 | 2.88ns |
| Metallo. | 1.9 ± 0.37 | 1.4 ± 0.17 | 1.8 ± 0.45 | 1.7 ± 0.35 | 0.37ns |
| Cyt. P450 | 13.3^{a} ± 0.2 | 15.3^{a} ± 0.59 | 14.5^{a} ± 1.06 | 18.3^{b} ± 0.50 | 10.1*** |

Table 6. Summary of biomarker responses to atrazine stress in grass shrimp (Palaeomonetes pugio). Treatments were 0 g/L (control), 1 g/L (low), 500 g/L (medium), and 1 mg/L (high) of atrazine. Table entries and units of measurement are as in Table 3.

Responses to heat stress: Seven biomarkers contributed to the significant multivariate response to heat stress (Table 3). Each was lowest in the control, intermediate in the low heat stress treatment, and highest in the medium heat stress treatment. Animals subjected to the 41° C. treatment did not survive. Ubiquitin was the only parameter that varied significantly among all treatments. Glutathione, LPO and B-crystallin were significantly higher in the low and medium heat stress treatments than in the control. Hsp60, Hsp70 and Mn SOD were significantly higher in the high heat stress treatment than in the control, with mean levels in the low heat stress treatment intermediate and statistically indistinguishable from those of the other treatments. Levels of metallothionine and cytochrome P450 did not vary significantly among treatments (Table 3).

Responses to heavy metal stress: Levels of seven biomarkers differed significantly among cadmium chloride treatments (Table 4). All biomarkers except Hsp70 were lowest in the control, and responded in a dose-dependent manner in the low, medium and high cadmium chloride treatments. In contrast, mean Hsp70 levels were lowest in the control, higher in the medium CdCl₂ treatment, slightly higher in the low CdCl₂ treatment, and highest in the high CdCl₂ treatment, which differed significantly from all other biomarkers (Table 4). Mean separation procedures revealed as few as two (GSH, Hsp70) and as many as three (LPO) statistically different and non-overlapping groups. Metallothionine was the most sensitive parameter, with more than a six-fold increase in mean levels at high CdCl₂ concentrations compared to the control. Mean levels of cytochrome P450 and Mn SOD did not differ significantly among treatments (Table 4).

Responses to fuel stress: Six biomarkers contributed to the significant multivariate response to bunker fuel stress (Table 5a). Three (GSH, Hsp60, and Mn SOD) were lowest in the control, and had progressively higher levels in the low, medium and high bunker fuel treatments. Cytochrome P450 was lowest in the control, higher in the low, and high bunker fuel treatement and highest in the medium treatment. Two biomarkers (ubiquitin and LPO) were lowest in the control, higher in the medium, slightly higher still in the low, and highest in the high bunker fuel treatment. Glutathione and Hsp60 were the most sensitive parameters, with highly significant 4.3 and 5.0-fold increases in mean levels at high bunker fuel concentrations compared to the control. Mean levels of Hsp70, B-crystallin and metallothionine did not vary significantly among bunker fuel treatments (Table 5a).

Seven biomarkers contributed to the significant multivariate response to diesel fuel stress (Table 5b). Five (LPO, ubiquitin, Hsp70, B-crystallin, and cytochrome P450) were lowest in the control, and had progressively higher levels in the low, medium and high bunker fuel treatments. Glutathione was lowest in the control, higher in the medium, higher still in the low, and highest in the high diesel fuel treatment. Mn SOD was lowest in the control, intermediate in the low and high treatments, and highest in the medium bunker fuel treatment. Three parameters were very sensitive to diesel fuel stress: GSH, Hsp70 and cytochrome P450 had highly significant 6.5, 6.0, and 3.9-fold increases from control to high diesel fuel treatments. Levels of metallothionine and Hsp60 did not vary significantly among diesel fuel treatments (Table 5b).

Responses to atrazine stress: Five biomarkers (GSH, LPO, Hsp60& 70, and cytochrome P450) contributed to the significant multivariate response to atrazine stress (Table 6). Lipid peroxide levels fell significantly in the presence of atrazine, compared to controls without it. Levels of Hsp70 increased in direct proportion to atrazine concentration, while GSH, Hsp60 and cytochrome P450 had more complex responses, with only the highest concentration of atrazine producing biomarker levels signficantly different from the controls. Two parameters were very sensitive to atrazine stress. Levels of LPO decreased 38%, while levels of Hsp60increased nearly five-fold from control to high atrazine treatments. Levels of ubiquitin, B-crystallin, SOD, and metallothionine did not vary significantly among atrazine treatments (Table 6).

Heat Stress: Heat stress is known to stimulate a concomitant induction of oxidative stress (Halliwell et al. (1999), Free Radicals in Biology and Medicine, 3^{rd} Edition, Oxford Science Publications, Oxford, England). This phenomenon is thought to be a result from heat-induced conformational changes in a number of enzymes that foster increased reactive oxygen species (ROS) production. Examples are aconitase's heat-induced ROS generation via Fenton chemistry from its iron-sulfur cluster or heat-induced electron transfer to O₂ instead of ubiquinone reduction by NADH:ubiquinone oxidoreductase (ibid.). The data above indicates that heat stress in grass shrimp induces oxidative stress, since LPO levels were significantly higher in heat-stressed samples than controls. GSH levels also increased in response to heat stress, suggesting not only a response to oxidative stress, but an extremely fast compensatory response. Levels of Mn SOD also were significantly elevated by heat stress, indicating that mitochondria were experiencing and responding to an oxidative stress. Increased levels of LPO and ubiquitin suggest that a major insult to cell structure integrity occurred during heat stress. Increased LPO levels during or following a stress event would also indicate a decline in functional lipid levels (Duthie (1993), “Lipid Peroxidation,” Eur. J. Clin. Nutr. 47:759-764), while an increase in ubiquitin levels indicates increased protein turnover. This interpretation is supported by the significantly higher levels of chaperone accumulation (Hsp60 and Hsp70) in response to heat stress when compared to control conditions, indicating increased protein denaturation (Hayes et al. (1995), “Induction of a 70-kD Heat Shock Protein in Scleractinian Corals by Elevated Temperature: Significance For Coral Bleaching,” Mol. Mar. Biol. Biotech. 4:36-42; Iwai (1999), “Roles of the Ubiquitin System in Stress Response,” Tanpakushitsu Kakusan Kuso 44:759-765). Induction of B-crystallin during heat stress suggests that the cytoskeletal integrity of shrimp was also being compromised. This small heat-shock protein is found only in the cytosol of animals and functions to protect cytoskeletal elements during stress. Metallothionein and cytochrome P450 2E1 did not respond significantly altered to heat stress. This indicates that shrimp were not stressed by either a xenobiotic or a heavy metal; more importantly, it shows these two markers are unresponsive to heat stress, which is not true for other cytochrome P450s (e.g. CYT P450 1E1).

Cadmium stress: Cadmium is detrimental to the cell because it inhibits some enzymes and generates oxidative stress via the Fenton reaction (Klassen (1999), supra). The above data indicated that an oxidative stress was occurring in grass shrimp exposed to cadmium chloride due to significantly higher LPO and Mn SOD levels. GSH levels also were significantly higher in exposed shrimp than in controls, again reflecting the peculiar response of GSH in heat-stressed shrimp (Dolphin et al. (1989), Glutathione. Chemical, Biochemical, and Medical Aspects, Vols. A and B, J. Wiley and Sons, New York, N.Y., U.S.A.). Cell structure integrity was also under assault as indicated by high LPO levels, significantly higher levels of ubiquitin than in controls, and significant accumulation of chaperones in cadmium-exposed shrimp compared to controls. B-crystallin homologue was also significantly higher in cadmium-exposed shrimp than controls indicating that cadmium has an adverse affect upon cytoskeletal elements. Induction of several classes of metallothionein proteins is a well-described response and adaptation to heavy-metal exposure (Klassen (1999), supra). Metallothionein levels were significantly higher in exposed shrimp than in controls, indicating both a specific response to heavy-metal exposure and induction of a compensatory action against its toxicity (Klassen (1999), supra; Masters et al. (1994), “Targeted Disruption of Metallothionein I and II Genes Increases Sensitivity to Cadmium,” Proc. Natl. Acad. Sci. USA 91:584-588). Cytochrome P450 2E1 was not induced by cadmium exposure, again indicating that it was not elicited by protein denaturing stress or an oxidative stress.

Diesel and Bunker Fuels: The water-accommodating fraction (WAF) of diesel fuel and bunker fuels elicited a cytochrome P450 2E1 response, indicating that this parameter is an appropriate indicator for diesel and bunker fuel exposure. Cell structure integrity was compromised by diesel fuel exposure as illustrated by the significant accumulation of LPO and ubiquitin. Diesel fuel did not induce significant accumulation of Hsp60, but significantly increased Hsp70 levels. Conversely, bunker fuel elicited a significant response for Hsp60, but not for Hsp70. Exposure to the WAF of diesel and bunker fuel induced an oxidative stress, which was detected as a significant induction of LPO and Mn SOD. GSH levels were significantly higher in both WAF of diesel and bunker fuel exposed samples.

Atrazine: Atrazine-exposed shrimp gave a clear indication of a xenobiotic exposure, as cytochrome P450 2E1 levels were significantly higher than in controls. In mammals, cytochrome P450 2E1 is instrumental in the catabolism of atrazine (Hanioka et al. (1998), “In Vitro Biotransformation of Atrazine by Rat Liver Microsomal Cytochrome P450 Enzymes,” Chem. Biol. Interact. 116:181-198; Hanioka et al. (1999), “In Vitro Metabolism of Simazine, Atrazine, and Propazine by Hepatic Cytochrome P450 Enzymes of Rat, Mouse and Guinea Pig, and Oestrogenic Activity of Chlorotriazines and Their Main Metabolites,” Xenobiotica 29:1213-1226). In concordance with a xenobiotic response, GSH levels were significantly higher in atrazine-exposed shrimp than in controls, indicating that GSH plays a role in the detoxification of atrazine (Kramer et al. (1988), “Role of the Glutathione Redox Cycle in Acquired and De Novo Multidrug Resistance,” Science 241:694-697).

### EXAMPLE 3

### Using Multiple Biomarkers to Assess the Health of Gastropods Exposed to Natural and Anthropogenic Stressors

A molecular biomarker system of the invention was used to assay specific parameters of a snail cell that are indicative of a non-stressed or stressed physiological condition. These cellular parameters are: lipid peroxide levels (LPO); total glutathione (GSH); heat-shock protein 60 (Hsp60); heat-shock protein 70 (Hsp70); B-crystallin homologue; small heat-shock protein 22 (Hsp22); small heat-shock protein 26 (Hsp26); manganese superoxide dismutase (Mn SOD); cytochrome P450 2E1 homologue; metallothionein class I; and ubiquitin. As in the previous examples, these parameters were chosen because they represent specific cellular physiological functions.

The eastern mudsnail, Ilyanassa obsoleta (Say), was selected as a model gastopod indicator species since it is a common and conspicuous element of the intertidal fauna from Canada to northern Florida. The snails are nonspecific grazers on organic deposits, benthic algae, epiphytes, and even scavenge carrion (Feller (1984), “Dietary Immunoassay of Ilyanassa Obsoleta, the Eastern Mud Snail,” Biol. Bull. Mar. Biol. Lab. Woods Hole 166:96-102; Curtis (1985), “The Influence of Sex and Trematode Parasites on Carrion Response of the Estuarine Snail Ilyanassa Obsoleta,” Biol. Bull. Mar. Biol. Lab. Woods Hole 169:377-390; Cranford (1988), “Behaviour and Ecological Importance of a Mud Snail (Ilyanassa Obsoleta) Population in a Temperate Macrotidal Estuary,” Can. J. Zool. 66:459-466; Kinlan et al. (1997), “Control of Periphyton on Zostera Marina By the Eastern Mudsnail, Ilyanassa Obsoleta,” Biol. Bull. Mar. Bio. Lab. Woods Hole 193:286-287). They reach highest densities on open mud flats with low flow, burrowing to escape dislodgement by the tides (Levinton et al. (1995), “The Effect of Water Flow on Movement, Burrowing, and Distributions of the Gastropod Ilyanassa Obsoleta,” Mar. Biol. 122:417-424). Ilyanassa populations are perennial, with individual life spans estimated to reach 30-40 years (Curtis (1995), supra). Egg capsules, laid on shells and other hard substrates, hatch to planktonic larvae that metamorphose and settle after several weeks (Richmond & Woodin, 1996; Froggett & Leise, 1999). The phenomenon of “imposex,” the imposition of male characteristics on female gastropods, has made Ilyanassa populations useful indicators of organotin pollution (Curtis (1994), “A Decade-Long Perspective on a Bioindicator of Pollution: Imposex in Ilyanassa Obsoleta on Cap Henlopen, Delaware Bay,” Mar. Environ. Res. 38:291-302; Oberdörster et al. (1998), “Testosterone Metabolism in Imposex and Normal Ilyanassa Obsoleta,” Mar. Pollut. Bull. 36:144-151).

Materials and Methods:

All chemicals for buffered solutions were obtained from Sigma Chemicals Co. (St. Louis, Mo., U.S.A.). Cadmium chloride was obtained from Sigma. Endosulfan and Atrazine were obtained from Supelco Chemicals (Bellefonte, Pa., U.S.A). Diesel and bunker fuel #2 were generous gifts from Exxon (Charleston, S.C., USA). PVDF and nitrocellulose membranes were obtained from Millipore Corp. (Bedford, Mass., U.S.A.) Dot blot and gel electrophoresis equipment were obtained from Bio-Rad Corp. (San Diego, Calif., U.S.A.) GSH-420 glutathione assay kits (Cat. #21023) and LPO-560 assay kits (Cat.#21025) were obtained from Oxis International, Inc. (Portland, Oreg., U.S.A.). Antibodies against Hsp70 (Cat. #SPA822), Hsp60 (Cat. #SPA805), B-crystallin (Cat. #SPA224), and ubiquitin (Cat. #SPA200) were obtained from Stressgen Biotechnologies, Inc. (Victoria, British Columbia, Canada). Metallothionein antibodies against MT-Class I (Cat.#18-0133) was obtained from Zymed (South San Francisco, Calif., U.S.A.). Hsp22 and Hsp26 antibodies were generous gifts from other laboratories. Anti-rabbit and anti-mouse conjugated alkaline phosphatase antibodies were obtained from Promega (Madison, Wis., U.S.A.) Protein standards of Hsp70, Hsp60, B-crystallin were obtained from Stressgen Biotechnologies. Protein standards of ubiquitin, Cu/Zn SOD, Mn SOD, and metallothionein were obtained from Sigma. Antibody to Cytochrome P450 2E1 was obtained from Oxis International, Inc.

Collection and Growth of I. Obsoleta:

Ilyanassa obsoleta (mud snails) were collected in Wadmalaw River near Charleston, S.C., U.S.A. This site is considered an un-impacted reference site and is a long-term ecologically monitored site by both the South Carolina Department of Natural Resources and U.S. National Oceanic and Atmospheric Administration Ecotoxicology Program. Water and sediment sample analysis of this site are conducted at least on a yearly basis for a number of pesticides, herbicides, metal, polyaromatic hydrocarbons, and eutrophic parameters (US NOAA NOS ORCA 128; SCDNR & US NOAA NMFS Tidal Creek Project).

Snails were acclimated to the laboratory at least one month prior to stress challenges. Preliminary experiments were carried out to determine the optimal means of growing and handling snails to induce a minimal stress response during laboratory experiments (data not shown). Snails were grown at 22-24° C. with a 14-hour photoperiod in 80 liter tanks filled with filtered, brackish water (20 ppt salinity), the sides of the tanks were covered with black, light-impermeable cloth. Tanks were aerated with air stones. Snails were fed commercially available algal pellets. There were no significant differences in biomarkers used between snails immediately caught and frozen at the collection site and snails that had acclimated for three weeks under laboratory conditions (date not shown).

Stress Exposure:

Handling of I. obsoleta from culture tanks to dosing chambers induced a significant response for many of the biomarkers, which is normally the case for any experimental organism, such as grass shrimp (Oberdörster et al. (1999), “Benthic Community Structure and Biomarker Induction in Grass Shrimp In an Estuarine System,” Arch. Environ. Contam. Toxicol. 37:512-518). Preliminary experiments demonstrated that the best method for reducing background levels of stress is to acclimate the snails in their dosing chambers seven days in advance of the stress challenge (data not shown). This was accomplished for each stress challenge, regardless of the stressor, by placing one snail (110-140 mg wet weight) in 1 L glass dosing chambers filled with 400 mL of filtered, brackish water (20 ppt salinity). Snails were held in these dosing chambers for 6 days before the beginning of the stress challenges. 300 mL of the brackish water in the dosing chamber was changed with fresh brackish water on the 2^{nd}, 4^{th}, and 6^{th} day of dosing chamber acclimation. Snails were fed algal pellets on the 1^{st}, 3^{rd}, and 5^{th} day of dosing chamber acclimation. Chambers were substrate free and were aerated with air stones while oxygen content and temperature were continuously monitored during the seven-day chamber acclimation. Ammonia content was measured on day 1 and day 6 of the seven-day chamber acclimation. During the seven-day acclimation, snails were subject to a 25 mol m⁻² s⁻¹ photosynthetic-active-radiation 14-hour photoperiod at 22° C. Dosing chambers were labeled for the type and dose of the stress with five replicates per treatment. Dosing chambers were then arranged in a randomized block design for stress challenging (Sokal and Rohlf, 1995). At the end of each 8-hour challenge, snails were immediately collected and frozen in liquid nitrogen and kept at −80° C. until sample preparation.

Heat stress—Snails were subject to an 8-hour temperature exposure either of 22° C. or 38° C. Temperature was ramped from 22° C. to 38° C. over a 70-minute period.

Cadmium exposure—Snails were subjected to an 8-hour exposure of 0 M, 5 M or 50 M cadmium chloride. The solvent carrier was water.

Atrazine exposure—Snails were subjected to an 8-hour exposure of 1 g/L or 1 mg/L acetone stock of technical grade atrazine (99%). Control animals were exposed to 50 L of acetone/400 mL brackish water, which was equal to the highest volume of atrazine solution delivered to atrazine-challenged samples.

Endosulfan exposure—Snails were subjected to an 8-hour exposure of 50 ng/L, 500 ng/L, or 1 g/L acetone stock of technical grade endosulfan (99%). Control animals were exposed to 50 L of acetone/400 mL brackish water, which was equal to the highest volume of endosulfan solution delivered to atrazine-challenged samples.

Bunker fuel #2 exposure—Snails were subjected to an 8-hour exposure of 0.5 g/L, 1 g/L, and 3 g/L water-accommodating fraction of bunker fuel. Water-accommodating fraction of bunker fuel was prepared according to Blenkisopp et al. (1996). 1 L of brackish water was added to Teflon-coated 1.1 L Nalgene bottles (Nalge Company, Rochester, N.Y., USA). The appropriate amount of bunker fuel was added to each bottle and allowed to mix, using a magnetic stirrer, for 48 hours. Brackish water was used as a control.

Sample Preparation and Assay:

Snails were cracked, the shell discarded, and examined for parasitism (Curtis (1997), “Ilyanassa Obsoleta (Gastropoda) As a Host for Trematodes in Delaware Estuaries,” J. Parasitol. 83:793-803) and imposex (Curtis (1994), “A Decade-Long Perspective on a Bioindicator of Pollution: Imposex in Ilyanassa Obsoleta on Cape Henlopen, Delaware Bay,” Mar. Environ. Res. 38:291-302). No evidence of either was found. The whole soft body was frozen in liquid nitrogen, ground frozen in a mortar and pestle, and then suspended in a solution consisting of 10 mM phosphate buffer (pH 7.8), 5 mM butylhydrotoluene, and 0.5% SDS. Samples were vortexed for 30 seconds and then centrifuged and supernatant placed in a new tube for sample analysis for LPO and GSH determination. Protein concentrations of samples were assayed by method of Ghosh et al. (1988), supra.

Samples were analyzed spectrophotometrically for LPO and total GSH content following the manufacture's instruction. Samples for each assay were analyzed in triplicate.

After performing LPO, GSH, and protein concentration assays, 100 L of a solution containing 20% SDS, 50 mM Tris-HCl (pH 7.8), 100 mM dithiothreitol, 80 mM EDTA, 3% polyvinyl pyrrolidone (w/v), 20 mM phenylmethylsulfonyl fluoride, 20 mM benzamide, 50 M -amino-caproic acid, and 1 g pepstatin A was added to 900 L of sample. Samples were then boiled for three minutes, allowed to sit at 25° C. for five minutes, and then centrifuged at 10,000× g for five minutes. Supernatant was transferred to a new tube and the pellet was discarded. Sample was then subjected to another protein concentration assay.

Dot Blot, Gel Electrophoresis, Immunoblotting, and Densitometric Analysis:

Samples were then assayed for Hsp70, Hsp60, B-crystallin, Hsp22, Hsp26, ubiquitin, cytochrome P450 2E1, Mn SOD, and metallothionein using immunochemical analysis. All samples were analyzed in triplicate. ELISPOT technique on nitrocellulose membrane was employed by using a dot blot apparatus. Western blotting on PVDF membrane was used after samples were subjected to SDS-PAGE. Both types of blots were blocked for 1 hour in either 5% non-fat-dried milk in 1×TBS (50 mM Tris/HCl (pH 9.8), 10 mM NaCl) or, for ubiquitin blots in 0.1% Tween-20 TBS solution. Blocking solution was decanted and blots were incubated in the appropriate primary antibody solution for 12 hours at 4° C. Primary antibody solution was decanted, blots subjected to four 10-minute washes in 1×TBS, and then incubated in the appropriate secondary antibody solution for one hour. Secondary antibody solution was decanted, blots were again subjected to four 10-minute washes in 1×TBS, then developed in a nitroblue tetrazolium/5-bromo-4-chloro-3-indolyl phosphate solution.

Once developed, blots were scanned into a computer and analyzed using NIH image software (Http://rsb.info.nih.gov/nih-image). A serial dilution of purified protein for each cellular parameter was included in each assay to allow sample quantification and assay quality control. For example, titered concentrations of purified metallothionein from rabbit were used as both a qualitative and quantitative standard for the samples assayed with metallothionein. Concentration standards for each assay were determined and a quadratic or polynomial equation was used to determine the concentration of each sample.

Because of the “uniqueness” of each of the cellular parameters, specific modifications were made for each ELISPOT assay. For example, ELISPOT analyses of metallothionein proteins followed a slightly modified protocol described by Mizzen et al. (1996), “Sensitive Detection of Metallothionein-1, -2, and -3 In Tissue Homogenates By Immunoblotting: A Method For Enhanced Membrane Transfer and Retention,” J. Biochem. Biophys. Methods 32:77-83. Small heat-shock proteins or B-crystallin analysis employed higher concentrations of SDS and DTT in the buffer and boiled for three minutes to prevent oligomerization of the sHsps under mild or non-denaturing conditions.

Statistical Analysis:

As in the preceding examples, multivariate analysis of variance (MANOVA) was used to test the null hypotheses that treatments had no significant effect on mean biomarker levels. When significant differences were found, univariate analyses (ANOVA) were used as in the preceding examples. Other aspects of the statistical analysis were similarly identical to those described with respect to the preceding examples.

Preliminary Experiments:

To determine that the antibodies bound to their specific targets, preliminary experiments were conducted to ensure the validity of the antibody assays. Snails were exposed either to high temperature, cadmium, ethanol, or acetone, homogenized in an SDS/50 mM dithiothreitol buffered solution, subjected to SDS-PAGE, immunoblotted to PVDF membrane, and assayed with at least 3 different antibodies per cellular parameter. Antibodies finally chosen as the enzyme immunoassay (EIA) had the best signal to noise ratio for each cellular parameter. Purified protein of each cellular parameter was run beside control and treated samples. The Hsp22 antibody crossreacted with an ca. 20-22 kDa protein that was heat inducible. The Hsp26 antibody crossreacted with an ca. 25-28 kDa protein that also heat inducible. Cytochrome P450 2E1 cross-reacted with an ethanol- and acetone-inducible proteins of ca. 50 kDa. This protein was detectable in controls and other treatments, but cadmium and heat stress did not elicit significant accumulation of this protein (data not shown). Hsp60 antibodies cross-reacted with one protein band of about 66 kDa that was inducible in all treatments (data not shown). The Hsp70 antibody chosen for this study cross-reacted with two to three proteins between ca. 70 kDa and 72 kDa. Hsp70 homologues were inducible by all treatments except ethanol and acetone. B-crystallin antibody cross-reacted with a single, ca. 19-21 kDa protein that was inducible by all treatments but undetectable in the control samples. This antibody does not cross-react with the sHsp22 or sHsp26 (p26) protein homologues of Artemia nor Drosophila sHsp26. Mn SOD antibody cross-reacted with a single protein of about 26 kDa that was inducible by both heat stress and cadmium, but not by exposure to acetone.

Treatments had significant effects on the vector of biomarker responses in the heat stress, atrazine, bunker fuel, and diesel fuel experiments (all Wilks'<0.026 and approximate F>2.25, all P<0.03; Table 7). Treatment means, their standard errors, and test statistics are reported in Tables 7-11.

TABLE 7
Heat Stress

| Biomarker | Control | Heat-stressed | F_{1,9} |
|---|---|---|---|
| GSH | 57 ± 2.0 | 34 ± 4.3 | 22.4* |
| LPO | 0.96 ± 0.051 | 1.32 ± 0.058 | 21.6* |
| Ubiquitin | 39 ± 2.5 | 105 ± 4.7 | 153.5*** |
| Hsp22 | 0 ± 0.2 | 24 ± 4.7 | 25.0ns |
| Hsp26 | 0 ± 0 | 41 ± 7.6 | 29.4ns |
| Hsp60 | 0.184 ± 0.0117 | 0.53 ± 0.0226 | 185.3*** |
| Hsp70 | 9.2 ± 1.02 | 36.4 ± 1.96 | 151.0*** |
| B-crystallin | 12 ± 3.5 | 35 ± 4.6 | 16.3* |
| Mn SOD | 14.2 ± 1.53 | 28.6 ± 2.98 | 18.5* |
| Metallo. | 19.2 ± 1.40 | 18.2 ± 2.13 | 0.2ns |
| Cyt. P450 | 9.6 ± 1.78 | 13.6 ± 2.54 | 1.7ns |

Table 7. Summary of biomarker responses to heat stress in mud snails (Ilyanassa obsoleta). Treatments were 22 ° C. (control) and 38 ° C. (heat-stressed). Entries in the table give treatment means ± 1 SE, F-statistics from a Welch (Hsp 22 and Hsp 26) or one-way ANOVA (all other biomarkers), and significance levels;
ns = not statistically significant ( adjusted for multiple tests; see text),
*P < 0.00465,
**P < 0.001,
***P < 0.0001.
Units: mM GSH/ g total protein; mM LPO/mg total protein; ng ubiquitin/ g total protein; pg Hsp or B-crystallin/ g total protein; relative protein concentrations for SOD, and cytochrome P450; ng metallothionein/mg total protein.

TABLE 8
Cadmium Chloride Stress

| Biomarker | Control | Low | High | F_{2,12} |
|---|---|---|---|---|
| GSH | 75^{a} ± 2.9 | 64^{b} ± 2.4 | 60^{b} ± 2.8 | 8.7* |
| LPO | 0.92 ± 0.058 | 1.1 ± | 1.16 ± 0.093 | 2.3ns |
| | | 0.089 | | |
| ubiquitin | 30^{a} ± 1.9 | 40^{ab} ± 2.8 | 50^{b} ± 5.0 | 8.1** |
| Hsp22 | 2^{a} ± 1.3 | 31^{b} ± 5.9 | 64^{c} ± 11.0 | 18.1** |
| Hsp26 | 14^{a} ± 3.5 | 32^{b} ± 4.1 | 58^{c} ± 5.3 | 26.2** |
| Hsp60 | 0.145^{a} ± | 0.419^{b} ± | 0.509^{b} ± 0.0558 | 27.8*** |
| | 0.0054 | 0.0271 | | |
| Hsp70 | 14.0^{a} ± 2.63 | 66.4^{b} ± 12.32 | 72.8^{b} ± 7.24 | 14.8** |
| B- | 18^{a} ± 2.7 | 54^{b} ± 9.6 | 76^{b} ± 6.4 | 18.3** |
| crystallin | | | | |
| Mn SOD | 28.0^{a} ± 1.00 | 58.2^{b} ± 5.30 | 61.8^{b} ± 4.14 | 22.3*** |
| Metallo. | 14.0^{a} ± 2.00 | 52.0^{b} ± 4.48 | 70.2^{c} ± 5.56 | 44.8*** |
| Cyt. P450 | 28.0 ± 2.55 | 28.6 ± 3.11 | 27.4 ± 3.14 | 0.04ns |

Table 9. Summary of biomarker responses to cadmium chloride stress in mud snails (Ilyanassa obsoleta). Treatments were 0 M (control), 5 M (low), or 50 M (high) cadmium chloride. Entries in the table give treatment means ± 1 SE, F-statistics from a Welch (Hsp 70 and Mn SOD) or one-way ANOVA (all other biomarkers), and significance levels;
ns = not statistically significant ( adjusted for multiple tests; see text),
*P < 0.00465,
**P < 0.001,
***P < 0.0001.
Treatment means with different superscript letters differed significantly. Units as in Table 7.

TABLE 9
Bunker Fuel Stress

| Biomarker | Control | Low | High | F_{2,12} |
|---|---|---|---|---|
| GSH | 68^{a} ± 5.5 | 48^{b} ± 2.0 | 43^{b} ± 4.5 | 9.3* |
| LPO | 0.88^{a} ± 0.073 | 1.34^{b} ± 0.108 | 1.34^{b} ± 0.081 | 9.0* |
| ubiquitin | 20^{a} ± 2.9 | 33^{b} ± 2.7 | 35^{b} ± 3.9 | 6.6* |
| Hsp22 | 2^{a} ± 2.0 | 27^{b} ± 7.7 | 40^{b} ± 11.7 | 21.5*** |
| Hsp26 | 7 ± 3.2 | 26 ± 8.2 | 41 ± 11.3 | 4.1ns |
| Hsp60 | 0.144 ± | 0.286 ± | 0.358 ± 0.0802 | 3.8ns |
| | 0.0206 | 0.0500 | | |
| Hsp70 | 6.6 ± 1.54 | 34.6 ± 7.17 | 35.6 ± 8.43 | 6.5ns |
| B- | 22 ± 6.2 | 44 ± 3.2 | 49 ± 6.6 | 6.9ns |
| crystallin | | | | |
| Mn SOD | 13.0^{a} ± 3.30 | 47.4^{b} ± 2.68 | 36.6^{c} ± 2.48 | 38.3*** |
| Metallo. | 6.6 ± 1.66 | 8.4 ± 1.83 | 8.8 ± 1.36 | 0.5ns |
| Cyt. P450 | 15.6 ± 4.86 | 35.4 ± 7.30 | 37.2 ± 4.57 | 4.4ns |

Summary of biomarker responses to bunker fuel stress in mud snails (Ilyanassa obsoleta). Treatments were 0 g/L (control), 0.5 g/L (low), 1 g/L (medium - not shown), and 3 g/L (high) of water-accommodating fraction of bunker fuel. Values for Hsp22 were log₁₀ (x + 1) transformed to meet the normality assumptions of parametric analyses. Table entries and units of measurement are as in Table 8.

TABLE 10
Atrazine Stress

| Biomarker | Control | Low | High | F_{2,12} |
|---|---|---|---|---|
| GSH | 88 ± 2.8 | 85 ± 3.6 | 83 ± 3.1 | 0.8ns |
| LPO | 1.10 ± 0.071 | 1.16 ± 0.040 | 1.32 ± 0.037 | 5.5ns |
| ubiquitin | 27 ± 2.3 | 42 ± 3.9 | 41 ± 3.7 | 6.2ns |
| Hsp22 | 2 ± 1.5 | 6 ± 2.0 | 6 ± 1.9 | 0.9ns |
| Hsp26 | 9^{a} ± 5.2 | 35^{b} ± 3.1 | 35^{b} ± 1.7 | 12.6* |
| Hsp60 | 0.252 ± | 0.238 ± | 0.158 ± 0.0086 | 0.8ns |
| | 0.0828 | 0.0883 | | |
| Hsp70 | 125.0 ± 5.96 | 123.4 ± 7.05 | 121.8 ± 6.08 | 0.02ns |
| B- | 17 ± 2.2 | 29 ± 1.3 | 25 ± 2.1 | 8.7ns |
| crystallin | | | | |
| Mn SOD | 6.4 ± 2.50 | 9.0 ± 1.76 | 7.8 ± 1.66 | 0.3ns |
| Metallo | 13.8 ± 4.09 | 18.2 ± 2.06 | 8.6 ± 1.72 | 3.9ns |
| Cyt. P450 | 5.8 ± 2.13 | 18.0 ± 2.90 | 17.8 ± 3.07 | 7.0ns |

Table 10. Summary of biomarker responses to atrazine stress in mud snails (Ilyanassa obsoleta). Treatments were 0 g/L (control), 1 g/L (low), or 1 mg/L (high) of acetone stock of technical grade atrazine (99%). Table entries and units of measurement are as in Table 8.

TABLE 11
Endosulfan Stress

| Biomarker | Control | Low | Medium | High | Test statistic |
|---|---|---|---|---|---|
| GSH | 62^{a} ± 8.0 | 48^{ab} ± 3.1 | 37^{bc} ± 2.3 | 29^{c} ± 2.035 | 12.0** |
| LPO | 0.98^{a} ± 0.049 | 1.08^{a} ± 0.049 | 1.60^{b} ± 0.084 | 1.80^{b} ± 0.089 | 29.2*** |
| ubiquitin | 44^{a} ± 2.786 | 44^{a} ± 3.8 | 50^{a} ± 3.22 | 26^{b} ± 3.78 | 9.4** |
| Hsp22 | 1^{a} ± 1.4 | 11^{a} ± 3.9 | 34^{b} ± 8.9 | 39^{b} ± 5.418 | 9.0* |
| Hsp26 | 0 ± 0 | 20 ± 12.673 | 19 ± 5.9 | 23 ± 3.3 | 10.6ns |
| Hsp60 | 0.172^{a} ± 0.0124 | 0.238^{a} ± 0.0302 | 0.396^{b} ± 0.0194 | 0.232^{a} ± 0.0384 | 11.7*** |
| Hsp70 | 6.6^{a} ± 1.86 | 15.4^{b} ± 2.38 | 23.2^{b} ± 1.20 | 15.2^{b} ± 2.31 | 12.7*** |
| B-crystallin | 16 ± 3.2 | 12 ± 3.1 | 11 ± 3.9 | 13 ± 2.7 | 1.0ns |
| Mn SOD | 3.2^{a} ± 2.27 | 26.2^{b} ± 4.22 | 29.2^{b} ± 6.95 | 33.8^{b} ± 3.18 | 7.0* |
| Metallo. | 17.8 ± 3.20 | 19.4 ± 1.99 | 22.2 ± 3.15 | 14 ± 3.11 | 1.3ns |
| Cyt. P450 | 8.2^{a} ± 5.96 | 8.6^{a} ± 2.36 | 36.2^{b} ± 8.78 | 45.6^{b} ± 2.80 | 29.2*** |

Table 11. Summary of biomarker responses to endosulfan stress in mud snails (Ilyanassa obsoleta). Treatments were 0 ng/L (control), 50 ng/L (low), 500 ng/L (medium), or 1 g/L (high) acetone stock of technical grade endosulfan (99%). Table entries and units of measurement are as in Table 8. Test statistics are X²₍₃₎ from a nonparametric Kruskal-Wallis Test (Hsp 26), or F_{3,15} from a Welch (Hsp 22) or one-way ANOVA (all remaining biomarkers).

Results:

Responses to Heat Stress: Eight biomarkers contributed to the significant multivariate response to heat stress (Table 7). Each was lowest in the control and higher in the heat-stressed samples, except for GSH. GSH was significantly lower in the heat-stressed samples. There was not a statistically significant difference for levels of Hsp26 between control and heat-stressed samples. Levels of metallothionine and cytochrome P450 did not vary significantly among treatments (Table 7).

Responses to Cadmium Stress: Levels of nine biomarkers differed significantly among cadmium chloride treatments (Table 8). All biomarkers, except GSH, were lowest in the control, and responded in a dose-dependent manner in the low and high cadmium chloride treatments. In contrast, mean GSH levels were highest in the control, lower in the low CdCl₂ treatment and even lower in the high CdCl₂ treatment (Table 8). Metallothionine was the most sensitive marker, with more than a six-fold increase in mean levels at high CdCl₂ concentrations compared to the control. Mean levels of cytochrome P450 and LPO did not differ significantly among treatments.

Responses to Fuel Oil Stress: Levels of nine biomarkers differed significantly among bunker fuel treatments (Table 9). Again, levels of GSH were lower in exposed samples than in controls. Levels of LPO, Mn SOD, Hsp22, Hsp26, B-crystallin, and Hsp70 were significantly higher in fuel-exposed samples than in controls. Hsp60 levels were not statistically different in fuel-exposed samples compared to controls. Cytochrome P450 levels were over two fold higher in fuel-exposed snails when compared to controls.

Responses to Atrazine Stress: Only Hsp26 was significantly different among atrazine treatments (Table 10).

Responses to Endosulfan Stress: Nine biomarkers differed significantly among endosulfan treatments (Table 11). Levels of GSH were significantly lower in a dose-dependent manner in all three treatments of endosulfan. LPO levels were significantly elevated in a dose-dependent manner in response to increasing concentration of endosulfan. Ubiquitin levels were not significantly different from low endosulfan treatment and controls, but were higher in the medium endosulfan concentration compared to controls and lower in the high endosulfan concentration compared to controls. Both Hsp22 and Hsp26 levels were significantly higher in endosulfan-exposed samples compared to control samples. In contrast, B-crystallin levels were not significantly altered. Mn SOD levels were significantly higher in endosulfan-treated samples than in controls, but were not significantly different between endosulfan treatments. Metallothionein was not affected by endosulfan exposure. Cytochrome P450 levels were significantly higher in samples exposed to the medium and high concentrations of endosulfan, but cytochrome P450 levels in snails exposed to the low-endosulfan treatment were not significantly different from controls.

We claim:

1. A method for assessing the health of an ecosystem, comprising
(a) measuring the levels of a plurality of different physiological parameters of a non-mammalian organism selected from the group consisting of arthropods, molluscs, and fish living in the ecosystem, each of said physiological parameters corresponding to a single, specific cellular function;
(b) identifying each of the measured levels in step (a) as normal or abnormal, wherein a normal level is within a range associated with healthy organisms and an abnormal level is within a range associated with physiologically stressed organisms;
(c) determining from the results of step (b) whether the organism is healthy or physiologically stressed; and
(d) if the determination in step (c) is that the organism is physiologically stressed, using the results of step (b) to assess the type and degree of stress occurring in the ecosystem, wherein at least two of the different physiological parameters whose level is measured in step (a) are cellular concentrations of markers selected from the group consisting of glutathione peroxidase, glutathione transferase, mitochondrial Hsp60, mitochondrial Hsp70, mitochondrial sHsp, mitochondrial superoxide dismutase, cytosolic superoxide dismutase, ubiquitin, and P-glycoprotein.

2. The method of claim 1, wherein step (d) additionally comprises determining whether the organism has adapted in response to the stress occurring in the ecosystem.

3. The method of claim 1, wherein step (d) additionally comprises assessing the physiological impact of the stress on the organism.

4. The method of claim 3, further comprising (e) predicting the future health of the ecosystem based on the physiological impact of the stress on the organism.

5. The method of claim 1, wherein step (d) additionally comprises identifying at least one stressor in the ecosystem that is causing the organism to be physiologically stressed.

6. The method of claim 5, wherein the at least one stressor is selected from heat, light, chemical contamination, and combinations thereof.

7. The method of claim 6, wherein the stressor is chemical contamination.

8. The method of claim 7, wherein the chemical contamination is contamination with heavy metals.

9. The method of claim 7, wherein the chemical contamination is contamination with polyaromatic hydrocarbons.

10. The method of claim 7, wherein the chemical contamination is contamination with organic solvents.

11. The method of claim 7, wherein the chemical contamination is contamination with herbicides.

12. The method of claim 6, wherein the stressor is heat.

13. The method of claim 6, wherein the stressor is light.

14. The method of claim 1, wherein the organism is a dinoflagellate.

15. The method of claim 1, wherein the organism is an arthropod.

16. The method of claim 1, wherein the organism is a mollusc.
